(19) United States
(12) Patent Application Publication
(10) Pub. No.: US 2007/0122804 A1
(43) Pub. Date: May 31, 2007

(54) Combined exponential and linear amplification

(76) Inventor: Guoliang Fu, Oxford (GB)

Correspondence Address: MERCHANT & GOULD PC, P.O. BOX 2903, MINNEAPOLIS, MN, 55402-0903, US

(21) Appl. No.: 10/531,848

(22) PCT Filed: Oct. 29, 2003
(86) PCT No.: PCT/GB03/04794

§ 371 Date: May 25, 2006

Related U.S. Application Data
(60) Provisional application No. 60/421,765, filed on Oct. 29, 2002.

(30) Foreign Application Priority Data
Dec. 28, 2002 (GB) 0230238.8

(51) Int. Cl. C12Q 1/68; C07H 21/04

(52) U.S. Cl. 435/6; 536/24.3

(57) ABSTRACT

Methods and compositions are provided for sensitive detection and quantitation of nucleic acids. Methods and compositions are further provided for genotyping. Probes of the invention allow target initiated amplification for single stranded, double stranded polynucleotides and pyrophosphate (PPi). DNA enzyme mediated detection method is also provided for detecting single stranded end products.

### BACKGROUND OF THE INVENTION

This invention relates to the field of nucleic acid amplification and detection. More particularly, the invention provides methods, compositions and kits for amplifying (i.e., making multiple copies) nucleic acid molecules and for detecting amplified sequences, which involve target initiated nucleic acid polymerization, chain reaction cascade and DNA enzyme mediated detection.

A number of methods have been developed which permit the implementation of sensitive nucleic acid detection based on amplification. They fall into two classes, enabling either target or signal amplification. Target amplification methods include the polymerase chain reaction (PCR), ligase chain reaction. (LCR), self-sustained sequence replication (3 SR), nucleic acid sequence based amplification (NASBA), and strand displacement amplification (SDA). Signal amplification technologies include branched DNA (bDNA), hybrid capture, and cleavase (invader assay), and measure nucleic acid targets by amplification of a surrogate marker. Rolling circle amplification (RCA) is a method that performs either target or signal amplification. (Birkenneyer and Mushahwar, J. Virological Methods, 35:117-126 (1991); Landegren, Trends Genetics, 9:199-202 (1993); Schweitzer and Kingsmore, Current opinion in Biotechnology, 12 21-27 (2001)).

The PCR method remains the most widely used DNA amplification and quantitation method. However, PCR in general suffers from several limitations that are well-known in the art, such as the requirement of expensive thermal cyclers, easy contamination, difficulty of quantification, amplification with different efficiencies for different DNAs, and limited multiplexing.

Current technologies for quantitative profiling of mRNA/cDNA levels in biological samples involve the use of either cDNA arrays (Schena et al., Proc. Natl Acad. Sci. USA, 91:10614-10619 (1994)) or high density oligonucleotide arrays (Lockhart et al, Nature Biotechnology, 14:1675-1680 (1996)). In the case of the cDNA arrays by Schena et al, the detection of a single molecular species in each element of the array requires the presence of at least 100,000 bound target molecules. In the case of the DNA chip arrays used by Lockhart et al, the detection limit for hybridized RNA is of the order of 2000 molecules.

Single nucleotide polymorphisms (SNPs) are the foundation of powerful complex trait and pharmacogenomic study. The analysis of large number of SNPs, however, has emphasized a need for inexpensive SNP genotyping methods of commensurate simplicity, robustness, and scalability. In general, current methods require preamplification of genomic DNA, followed by SNP genotyping with an allele discrimination method, such as DNA cleavage, ligation, single base extension or hybridization. Current methods are limited either by expense, inaccuracy, consumption of sample DNA, or lack of scalability (Faruqi et al. BMC Genomics (2001) 2:4). Accordingly, there is a need for nucleic acid detection methods that are both sensitive and quantitative.

It is therefore an object of the disclosed invention to provide a method of detecting nucleic acid in low concentration.

It is another object of the disclosed invention to provide a method of determining the amount of specific target nucleic acid sequences present in a sample where the number of signals measured is proportional to the amount of a target sequence in a sample and where the ratio of signals measured for different target sequences substantially matches the ratio of the amount of the different target sequences present in the sample.

It is another object of the disclosed invention to provide a method of detecting the presence of target nucleic acid sequences representing individual alleles of a target genetic element.

It is another object of the disclosed invention to provide a method for high throughput SNP genotyping, detecting nucleotide methylation, and different gene splicing.

It is another object of the disclosed invention to provide a method of end product detection by DNA enzyme mediated cleavage of RNA or DNA-RNA chimera substrates.

### SUMMARY OF THE INVENTION

Disclosed are compositions and methods for amplifying and detecting nucleic acid. The methods of invention make the use of a specially designed oligonucleotide probe, referred to as “Amplification Repeat Templates” (ART) probe. Amplification is accomplished through combined exponential and linear amplification (CELA) which allows production of numerous copies of single stranded end product (SSEP), double stranded end product and pyrophosphate (PPi).

The ART probe molecules are single stranded or partially double stranded linear or circular nucleic acid which comprise: a target complementary portion, template portion(s), at least one enzyme acting portion, and with or without a 3′ end block portion. The ART probe may comprise a helper primer that makes some part(s) of the probe double stranded. The ART probe may comprise an antisense DNA enzyme or an antisense RNA enzyme. An ART probe may not comprise all portions and may comprise additional portions.

The enzyme acting portions may comprise a RNA polymerase promoter. The enzyme acting portions may comprise RNase H acting sequences. The enzyme acting portions may comprise a nuclease digestion site, which supports digesting an opposite strand of the probe when double stranded. The nuclease digestion site may comprise modified nucleotides, whereby the digestion site on the probe is resistant to nuclease cleavage and the opposite unmodified strand is sensitive to cleavage. The modified nucleotides may comprise phosphorothioate linkages.

The enzyme acting portions may comprise the combination of the RNase H acting sequences and the RNA polymerase promoter or the combination of the RNase H acting sequences and the nuclease digestion sites or the combination of the nuclease digestion sites and the RNA polymerase promoter or the combination of more than one of the nuclease digestion sites.

The nuclease digestion sites may comprise a restriction site having a restriction enzyme recognition sequence and a cleavage site. The restriction site may comprise a type IIS restriction enzyme site. It is preferred that the enzyme cleavage site of the type IIS restriction site is located on the target complementary portion. It is more preferred that for SNP genotyping, methylation analysis, and splicing analysis the type IIS restriction enzyme cleavage site corresponds to a SNP or mutation site, methylation nucleotide, or gene splicing site. The type IIS restriction site may be the Fok I site.

The probe may comprise helper primer(s), wherein the helper primer comprises at least one portion complementary or substantially complementary to a part of the probe. The helper primer may comprise a 3′ end blocking moiety, whereby the 3′ end of the helper primer is not extendible by a DNA polymerase. The helper primer may not comprise a 3′ end blocking moiety, whereby the 3′ end of the helper primer is extendible by a DNA polymerase.

The helper primer may comprise sequence complementary to the enzyme acting portion(s) or part of the enzyme acting portion(s) of the probe with or without flanking sequences, whereby hybridization between the helper primer and the probe makes the enzyme acting portion(s) double stranded or partially double stranded. The helper primer may comprise a 3′ end sequence which is extendable and is complementary to a sequence 3′ to one of the enzyme acting portions of the probe. The 3′ end sequence of the helper primer may have a length of 2 to 15 nucleotides, or preferably 3 to 10 nucleotides, or even preferably 4 to 8 nucleotides.

The helper primer may further comprise target complementary portion(s), wherein the target region(s) complementary to the helper primer is adjacent or substantially adjacent to the target region complementary to the probe. The helper primer may comprise 3′ and 5′ target complementary portions, wherein the target region complementary to the probe is located in the middle of the target regions complementary to the helper primer and is adjacent or substantially adjacent to the target regions complementary to the helper primer.

The target complementary portion of the probe may comprise a sequence complementary or substantially complementary to a target region of interest, wherein the target complementary portion of the probe hybridizes to the target region of interest and becomes double stranded, whereby one or more than one or part of the enzyme acting portion(s) of the probe is partially or fully functional.

The enzyme acting portion(s), the target complementary portion and the template portion(s) of the probe may overlap each other or may have one portion embedded in other portions.

The target complementary portion and/or the enzyme acting portion(s) and/or the template portion(s) of the probe may comprise modified nucleotides, whereby modified nucleotides are resistant to nuclease cleavage. In some embodiments, when the target is RNA and/or the single stranded end products (SSEP) are RNA, and RNase H is used in a reaction, it is preferred that the target complementary portion and/or enzyme acting portion and/or template portion comprise chimeric RNA and DNA. After annealing of target or SSEP RNA with the ART probe the double stranded RNA/RNA part is resistant to RNase H cleavage so that the target or SSEP RNA are not completely digested away, while the RNA on the RNA/DNA part is digested and the 3′ end of the digested RNA is used as an extension initiating site. It is also preferred that the RNA part on ART probes is modified so that it is not digested by any nuclease. The modified nucleotides may comprise phosphorothioate linkages.

The template portions of the probe may comprise two identical or nearly identical sequences in the same orientation, wherein the two identical or nearly identical sequences may be separated by at least one enzyme acting portion, which may comprise RNA polymerase promoter, or restriction enzyme site. The circular probe may comprise one template portion with other portions embedded in it.

In some embodiments, when DNA enzyme mediated detection is used, it is preferred that the template portions comprise a catalytically inactive antisense sequence complementary to a DNA enzyme. It is preferred that the DNA enzyme may be 10-23 or 8-17 DNAzymes.

The 3′ end block portion of the probe may be chemical moiety, which makes the 3′ end of the probe not extendible by a DNA polymerase. Any end of the probe and/or helper primer may be attached to a solid support.

One embodiment provides a method of detecting a target nucleic acid sequence or multiple target nucleic acid sequences of interest in a sample, the method comprising the steps of: (a) contacting probes or a set of probes with a nucleic acid sample under suitable hybridization conditions, wherein the target complementary portions of the probes or the target complementary portions of the probes and helper primers hybridize the target sequence(s) and become double stranded, whereby one or more than one or part of the enzyme acting portion(s) of the probe is partially or fully functional; (b) causing all enzyme acting portions of the probes to be double stranded and fully functional; (c) treating the probes containing double stranded enzyme acting portion(s) so as to produce the single stranded end product (SSEP); (d) annealing the SSEP to free probes and causing all enzyme acting portions of the probes to be double stranded and fully functional; (e) repeating steps of (c) and (d), whereby the probes are converted to double stranded or partially double stranded form, and multiple copies of the SSEP are produced repeatedly; and (f) detecting directly or indirectly the end products so produced: double stranded end product, SSEP and pyrophosphate (PPi). All steps of the method may be performed in a single reaction or in separated reactions.

In some embodiments, when the target nucleic acid is RNA and step (a) causes one of the enzyme acting portion, the RNase H digesting sites, to be double stranded and functional; wherein step (b) comprises: digesting the RNA strand by RNase H, extending the 3′ end of partially digested strand using the probe as template by a DNA polymerase, whereby all other enzyme acting portions on the probes become double stranded and functional. The other enzyme acting portions on the probes may comprise a restriction site or RNA polymerase promoter or both a restriction site and an RNA polymerase promoter.

In further embodiments, extending the 3′ end of partially digested strand may further comprise strand displacement by the DNA polymerase or other strand displacement factors.

In some embodiments, when one of the enzyme acting portions is a restriction site and is located on the target complementary portion of the probe and step (a) causes the restriction site to be double stranded and fully functional, step (b) comprises: digesting an opposite strand of the probes on the restriction site by a restriction enzyme, and extending the 3′ end of the digested strand using the probe as template by a DNA polymerase, whereby all other enzyme acting portions on the probes become double stranded and functional. The other enzyme acting portions on said probes may comprise other restriction site, RNA polymerase promoter or both restriction site and RNA polymerase promoter. Alternatively, said restriction site may be the only enzyme acting portion on the probe. In further embodiments, extending the 3′ end of the digested strand may further comprise strand displacement by the DNA polymerase or other strand displacement factors.

In some embodiments, when one of the enzyme acting portions is a type IIS restriction enzyme site with the cleavage site on a target complementary portion of the probe and the recognition site on either side of the target complementary portion of the probe, and step (a) causes the target complementary portions of the probe to be double stranded, whereby a functional cleavage site of the type IIS restriction site is formed, step (b) comprises: annealing helper primers to the probes and causing the recognition sequence of the type IIS restriction site double stranded. In one embodiment, annealing helper primers to the probes and causing the recognition sequence of the type IIS restriction site double stranded comprises: annealing the helper primers directly to the type IIS restriction enzyme recognition sequence with or without flanking sequences whereby a double stranded recognition sequence of the type IIS restriction site is formed. In another embodiment, annealing helper primers to the probes and causing the recognition sequence of the type IIS restriction site double stranded comprises: annealing the 3′ end sequence of the helper primer to a sequence 3′ of the type IIS restriction recognition sequence and extending the 3′ end sequence of the helper primer by a DNA polymerase using the probe as template, whereby a double stranded recognition sequence of the type IIS restriction site is formed.

In some embodiments, when the target complementary portions of the probes hybridize to free 3′ end(s) of the target sequence(s), step (b) comprises: extending the free 3′ end(s) of the target sequence(s) by a DNA polymerase using the probes as templates, whereby other enzyme acting portions on the probes become double stranded and functional.

In some embodiments, when the enzyme acting portions of the probe comprises a restriction site, step (c) comprises: digesting opposite strands of the probes on the restriction site by a restriction enzyme, extending the 3′ end of the digested strand by a DNA polymerase, and repeating the digesting and the extending, whereby multiple copies of SSEP DNA are produced. In further embodiments, extending the 3′ end of the digested strand may further comprise strand displacement by the DNA polymerase or other strand displacement factors.

In some embodiments, when the enzyme acting portions of the probe comprise RNA polymerase promoter, step (c) comprises: repeated transcription by the RNA polymerase acting on the RNA polymerase promoter, whereby multiple copies of SSEP RNA are produced.

In some embodiments, when the enzyme acting portions of the probe comprise both restriction site and RNA polymerase promoter, step (c) comprises: digesting opposite strands of the probes on the restriction site by a restriction enzyme, extending the 3′ end of digested strands by a DNA polymerase, repeating the digesting and the extending, whereby multiple copies of SSEP DNA are produced, and repeated transcription by the RNA polymerase acting on the RNA polymerase promoter, whereby multiple copies of SSEP RNA are produced. In further embodiments, extending the 3′ end of the digested strand may further comprise strand displacement by the DNA polymerase or other strand displacement factors.

In some embodiments, when the SSEP are DNA molecules or RNA molecules or both DNA and RNA molecules, step (d) comprises: annealing the SSEP to sequence portions of free probes and extending the 3′ ends of the SSEP using the free probes as templates, whereby all enzyme acting portions of the probes become double stranded and functional.

In some embodiments, when the SSEP are RNA molecules, step (d) comprises: annealing the SSEP to sequence portions of free probes, digesting the SSEP by RNase H, and extending the 3′ end of partially digested SSEP using the free probes as templates, whereby all enzyme acting portions become double stranded and functional.

In some embodiments, when the probes are circular molecules, the sequences of the SSEP RNA or DNA comprise one or more than one sequence unit that is complementary to the probes, step (d) comprises: annealing the SSEP to the whole or parts of the free probes, whereby the enzyme acting portions become double stranded and functional.

In some embodiments, when the template portions comprise an antisense DNA enzyme, the method produces multiple copies of single stranded functional sense DNA enzyme, step (f) detecting single stranded end product comprises: including an RNA or DNA-RNA chimeric reporter substrate in the reaction, wherein the RNA or DNA-RNA chimeric reporter substrate comprises fluorescence resonance energy transfer fluorophores incorporated on either side of a DNAzyme cleavage site, cleaving the reporter substrate by sense DNA enzyme, whereby cleavage of the reporter substrate produces an increase in fluorescence signal.

The invention also provides a kit for detecting a target nucleic acid sequence or multiple target nucleic acid sequences of interest. In an embodiment, the kit comprises: a set or sets of probes, helper primers, a detection substrate, restriction enzymes, RNA polymerase, RNase H, DNA polymerase, buffers, dNTPs, NTPs, other reagents and instructions.

### BRIEF DESCRIPTION OF THE DRAWINGS

FIG. 1 shows diagrams of examples of various Amplification Repeat Template (ART) probes. Target sequences are shown as indicated; various portions of probes, modified regions and helper primer are represented by various drawings as indicated in the first several diagrams of probes, which should be regarded as having similar meaning in the other probe diagrams.

FIG. 2 shows diagrams of examples of CELA reactions. CELA using linear probes is shown in FIG. 2A and CELA using circular probes is shown in FIG. 2B. A target nucleic acid is hybridized to target complementary portion of ART probes. The target strand on the ART probes is digested by a nuclease. The digested strand is extended by a DNA polymerase, therefore the downstream enzyme acting portions, if any, become double stranded. Following subsequent repeated polymerization, multiple copies of SSEP are generated, which then anneal to free ART probes, prime new polymerization, and generate new SSEP. The resulting end products, double stranded polynucleotides, single stranded SSEP and PPI, are then subjected to detection.

FIG. 3 shows diagrams of examples of various Amplification Repeat Template (ART) probes. Target RNA sequences and various portions of probes are shown as indicated.

FIG. 4 shows diagrams of examples of CELA reaction. CELA using linear probes is shown in FIG. 4A and CELA using circular probes is shown in FIG. 4B. A target RNA sequence is hybridized to the target complementary portion of ART probes. The RNA strand on the double stranded RNA/DNA hybrid is partially digested by RNase H. The partially digested strand is extended by a DNA polymerase, therefore the downstream enzyme acting portions, if any, become double stranded. Following subsequent repeated polymerization, multiple copies of SSEP are generated, which then anneal to free ART probes, prime new polymerization, and generate new SSEP. The resulting end products, double stranded polynucleotides, single stranded SSEP and PPI, are then subjected to detection.

FIG. 5 shows diagrams of examples of various ART probes. The ART probes comprise RNA polymerase promoter sequences.

FIG. 6 shows diagrams of examples of CELA reaction. CELA using linear probes is shown in FIG. 6A and CELA using circular probes is shown in FIG. 6B. A target RNA or DNA sequence is hybridized to the target complementary portions of ART probes; the ART probes comprise RNA polymerase promoter sequence. The target strand on the double stranded RNA/DNA or DNA/DNA hybrid is digested by an enzymatic digestion. A DNA polymerase extends the 3′ end of digested strand. Once the promoter sequences become double stranded, the RNA polymerase acts on the promoter and generates multiple copies of RNA transcripts, i.e. SSEP RNA sequences, which then anneal to free ART probes, prime new extension, and generate new SSEP. The resulting end products: double stranded polynucleotides, single stranded SSEP and PPI, are then subjected to detection.

FIG. 7 shows diagrams of examples of ART probes. The ART probes comprise type IIS restriction sites as one of the enzyme acting portions.

FIG. 8 shows diagrams of examples of CELA reactions for genotyping SNPs. CELA using linear probes is shown in FIG. SA and CELA using circular probes is shown in FIG. 8B. A target RNA or DNA sequence is allele-specifically hybridized to target complementary portions of ART probes, while helper-primer anneal to both ART probes and a target region, which is adjacent to the hybridization region of the ART probe. In FIG. 8A, the 3′ end of helper-primer is extended by a DNA polymerase using ART probe as template therefore double stranded functional type IIS restriction recognition site is created. In FIG. 8B, the double stranded functional type IIS restriction recognition sites are created by hybridization between helper primers and ART probes. The target strands on the double stranded target complementary portions of ART probes are digested by type IIS restriction enzyme (for example Fok I), while ART probe strands are resistant to cleavage due to modified nucleotides. The 3′ end of digested strands are extended by a DNA polymerase. Through subsequent repeated digestion and extension, multiple copies of SSEP are generated, which then anneal to free ART probes, prime new extension, digestion, and generate new SSEP. The resulting end products, double stranded polynucleotides, single stranded SSEP and PPI, are then subjected to detection.

FIG. 9 shows examples of detection methods.

FIG. 10 shows a diagram of an example of DNAzyme mediated detection of single stranded end products (SSEP). The template portion of ART probe comprises a complementary (antisense) sequence of a DNAzyme, for example 10-23 DNAzyme. During CELA reaction, SSEP are produced that contain active (sense) copies of DNAzymes. The DNA enzyme binds an RNA or DNA-RNA chimeric reporter substrate which contain fluorescence resonance energy transfer fluorophores incorporated on either side of a DNAzyme cleavage site. Cleavage of this reporter substrate produces an increase in fluorescence that is indicative of successful amplification of the target initiated single stranded SSEP.

FIG. 11 shows a diagram showing an ART probe sequence, its target sequence beta-actin gene, and the structures of reaction end products; the details are described in Example 1. The italicized bases are the HincII and NaeI recognition sites and the underlined bases are template sequences. “s” denote phosphorothioate linkage.

FIG. 12 shows results of gel electrophoresis of reaction products of Example 1.

FIG. 13 shows a diagram showing an ART probe sequence containing Fok I site, a helper primer sequence and target sequence; details are described in Example 2.

FIG. 14 shows a diagram showing an ART probe sequence containing T7 RNA polymerase promoter, and its target sequence; details are described in Example 3.

FIG. 15 shows a diagram showing a circular ART probe sequence, helper primer sequence, and target sequence; details are described in Example 4.

### DETAILED DESCRIPTION OF THE INVENTION

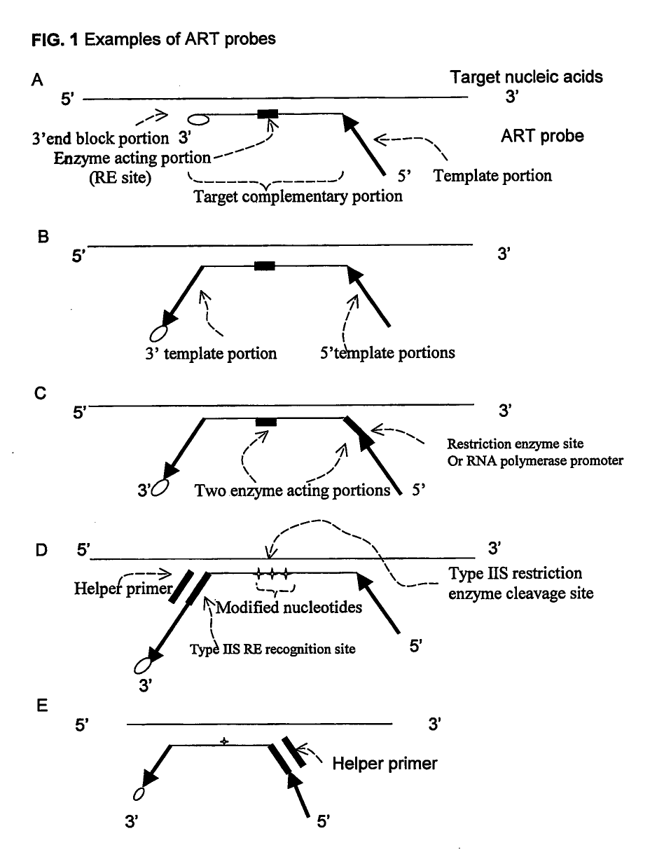

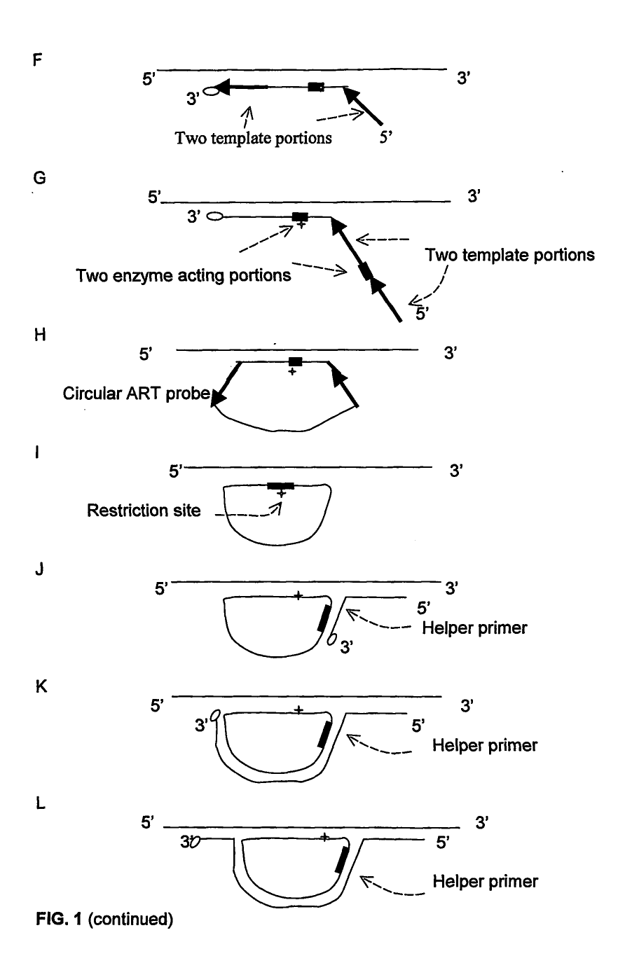

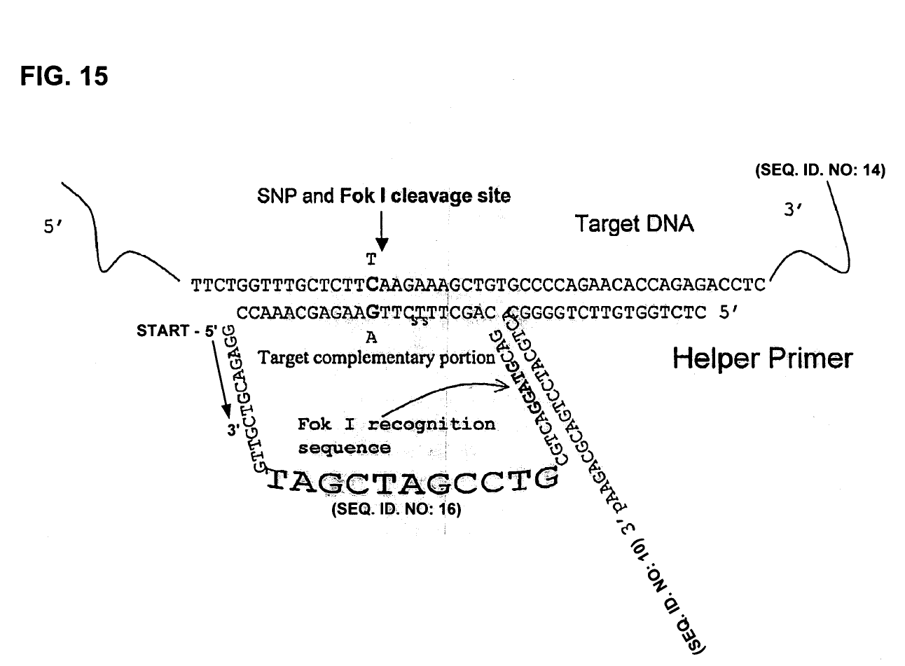

Examples of various probes are shown in FIGS. 1, 3, 5, 7, 11, 13, 14 and 15. They should not be regarded as limited, and any variant may be made without deviating from the spirit and scope of the invention.

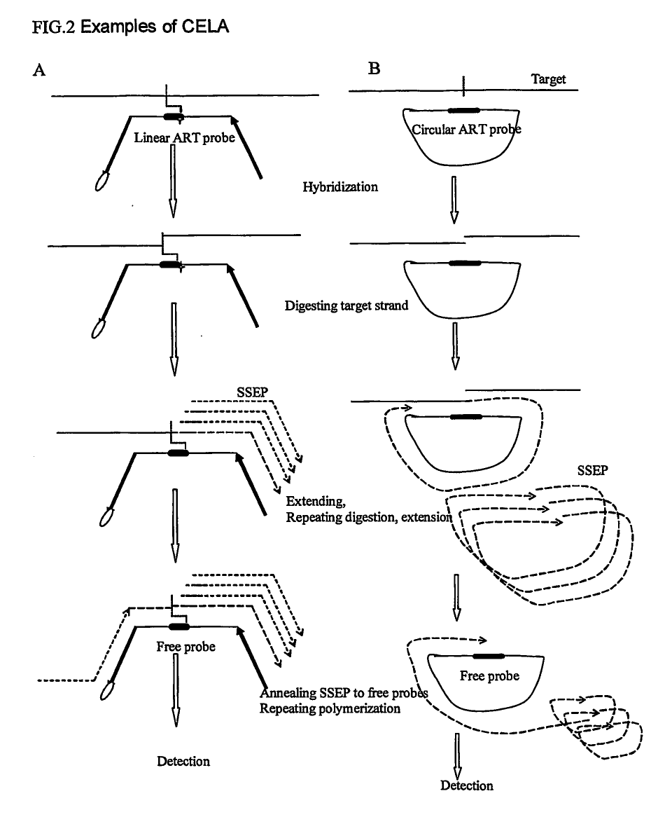

An example of a method of the invention is outlined below (FIG. 2).

A CELA reaction using linear probes is shown in FIG. 2A and a CELA reaction using circular probes is shown in FIG. 2B. A target nucleic acid is hybridized to the target complementary portion of the ART probes. An enzyme acting portion is located on the target complementary portion. The enzyme acting portion may be any nuclease digestion site which supports digesting an opposite strand of the probe when double stranded. In this example the nuclease digestion site is a restriction site and may be modified. Alternatively, the nuclease digestion site is a nicking restriction enzyme site where nucleotide modification on the probe is not required. The target strand on the ART probes is digested by the restriction enzyme. The 3′ ends of the digested strands are extended by a DNA polymerase, therefore the downstream enzyme acting portions, if any, become double stranded. The digestion and extension are repeated multiple times; the process generates multiple copies of single stranded end products (SSEP). In the example of the reaction shown in FIG. 2A, where the probes are linear molecules, the SSEP are complementary to the sequence 5′ to the restriction site of the probe that includes 5′ template portion sequence and part of the target complementary portion sequence of the probe. Because the 3′ template portion and 5′ template portion of the probe comprise identical or nearly identical sequences, the 3′ end of SSEP is complementary to 3′ template portion. The SSEP anneal to 3′ template portions of free probes and are extended by the DNA polymerase. Therefore double stranded ART probes are formed that trigger repeated digestion and extension. In the example of the reaction showing in FIG. 2B, where the probes are circular molecules, the SSEP are complementary to the whole sequence of the ART probe. The SSEP anneal fully or partially to free ART probes and are extended by the DNA polymerase, therefore double stranded ART probes are formed that trigger repeated digestion and extension. It is preferred that extension of the 3′ ends of the SSEP or the 3′ ends of the digested strands may be carried by the DNA polymerase having strand displacement activity or containing other strand displacement factors. The resulting end products, double stranded polynucleotides, SSEP and PPi, are then subjected to detection.

Because all reagents are in a single tube, all above steps may occur simultaneously. There is no distinct boundary between the steps. As long as there is free ART which is not hybridized to a SSEP, the amplification remains exponential. Once all ARTs hybridize to SSEP, the amplification may be linear. The reaction will accumulate double stranded polynucleotides, single stranded SSEP and PPi which can be labeled and detected.

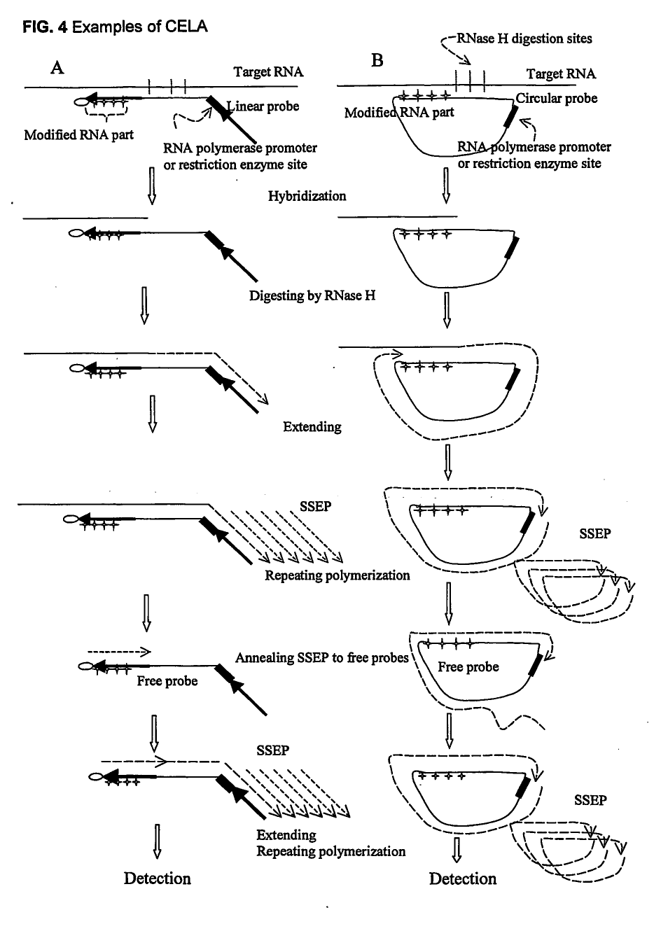

Another example of a method of the invention is outlined below (FIG. 4).

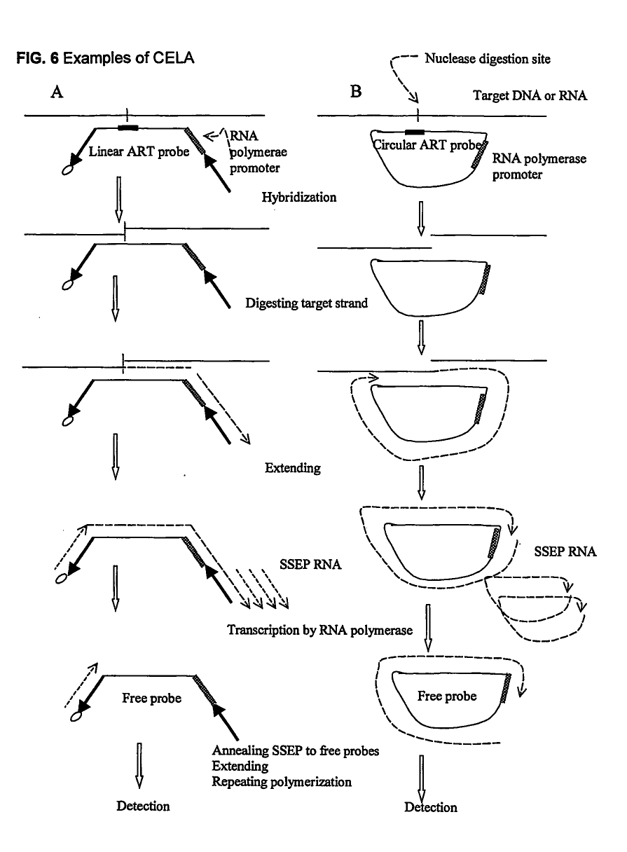

A CELA reaction using linear probes is shown in FIG. 4A and a CELA reaction using circular probes is shown in FIG. 4B. A target RNA sequence is hybridized to the target complementary portion of ART probes. In this example, the target complementary portion of the probe is also the enzyme acting portion—the RNase H digestion sites. The 3′ part of the target complementary portion is made by ribonucleotides which may comprise phosphorothioate linkages. The target RNA strand on the double stranded RNA/DNA hybrid is digested by RNase H, whereas the target RNA strand on the double stranded RNA/RNA hybrid is resistant to RNase H digestion. The 3′ end of the partially digested target RNA strand is extended by a DNA polymerase, therefore the downstream enzyme acting portions, if any, become double stranded. In this example, the downstream enzyme acting portions may comprise RNA polymerase promoter or restriction enzyme site. When the downstream enzyme acting portions is a restriction enzyme site, the next steps of the reaction are the same as described in the first example (FIG. 2). When the downstream enzyme acting portions is a RNA polynlerase promoter, the next steps of the reaction are the same as described in the next example (FIG. 6).

Another example of a method of the invention is outlined below (FIG. 6).

A CELA reaction using linear probes is shown in FIG. 6A and a CELA reaction using circular probes is shown in FIG. 6B. One of the enzyme acting portions of the ART probes comprise RNA polymerase promoter. A target RNA or DNA sequence is hybridized to the target complementary portions of ART probes. The target strand on the double stranded RNA/DNA or DNA/DNA hybrid of the ART probe is digested by a nuclease which may be a restriction enzyme or RNase H. The 3′ ends of digested a strand or a partially digested strand are extended by a DNA polymerase. Therefore the downstream enzyme acting portions, the RNA polymerase promoter, become double stranded and fully functional. The RNA polymerase acts on the promoter and generates multiple copies of RNA transcripts, i.e. the SSEP RNA sequences. In FIG. 6A, the probes are linear molecules, the SSEP RNA are complementary to the 5′ template portion sequence of the probe. Because the 3′ template portion and 5′ template portion of the probe comprise identical or nearly identical sequences, the 3′ end of SSEP is complementary to 3′ template portion. The SSEP anneal to 3′ template portions of free probes and are extended by the DNA polymerase, therefore double stranded ART probes are formed that trigger repeated transcription of SSEP RNA. In FIG. 2B, the probes are circular molecules; the SSEP may comprise one or more sequence units each of which is complementary to the whole sequence of the ART probe. The SSEP RNA anneal fully or partially to free ART probes and the 3′ ends of SSEP are extended by the DNA polymerase. Therefore double stranded ART probes are formed that trigger repeated transcription of SSEP RNA. Alternatively, the SSEP RNA anneal fully or partially to free ART probes, then are partially digested by RNase H, and the 3′ ends of partially digested SSEP are extended by the DNA polymerase, therefore polymerase. Therefore double stranded ART probes are formed that trigger repeated transcription of SSEP RNA. If the linear or circular ART probes comprise both restriction site and RNA polymerase promoter and a reaction includes the corresponding restriction enzyme and RNA polymerase, the reaction may produce both SSEP RNA and SSEP DNA that may then trigger repeated extension and digestion, and repeated transcription. It is preferred that extension of the 3′ ends of the SSEP or the 3′ ends of the digested strands may be carried by the DNA polymerase having strand displacement activity or containing other strand displacement factors. In the case of the circular ART probes that comprise both RNase Hacting sites and RNA polymerase promoter, the reaction includes the corresponding RNase Hand RNA polymerase. The SSEP RNA comprise one or more sequence units complementary to the whole sequence of the ART probe and anneal fully or partially to free ART probes. The SSEP RNA are partially digested by RNase H, and the 3′ ends of partially digested SSEP are extended by the DNA polymerase. If the DNA polymerase has strand displacement activity, the extension and strand displacement using circular probes as template may produce long single stranded end products that comprise multiple sequence units of which each is complementary to the whole sequence of the probe. The long single stranded end products anneal to free probes; therefore double stranded RNA polymerase promoter may be formed which trigger repeated transcription of SSEP RNA. The SSEP RNA then anneal to free ART probes and trigger flirther strand extension, displacement and transcription. The resulting end products include: double stranded end products, single stranded end products and PPi, which are then subjected to detection.

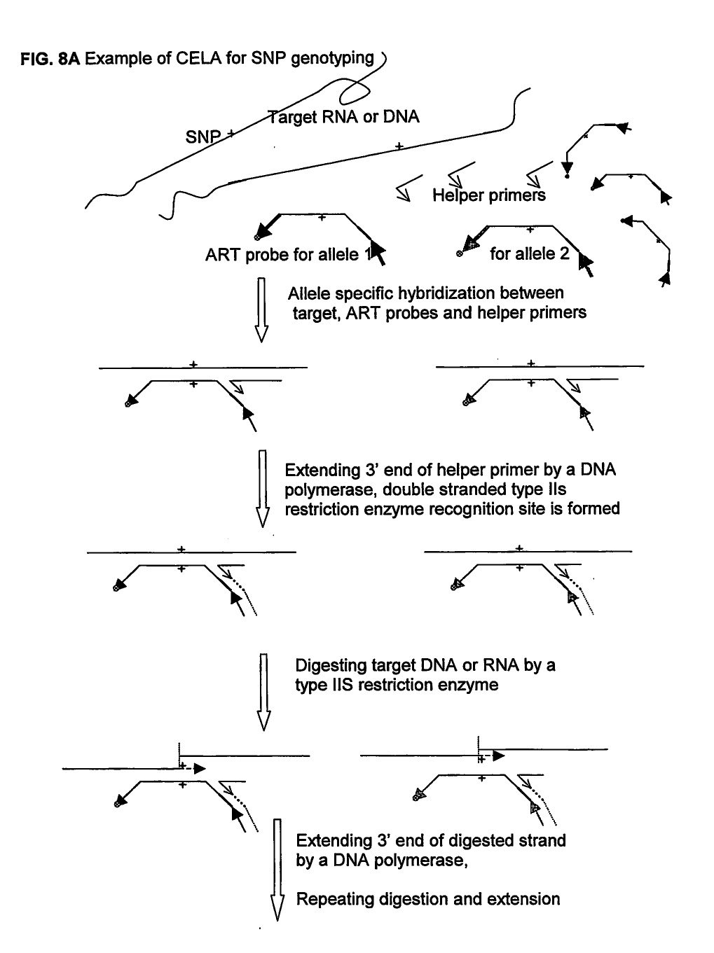

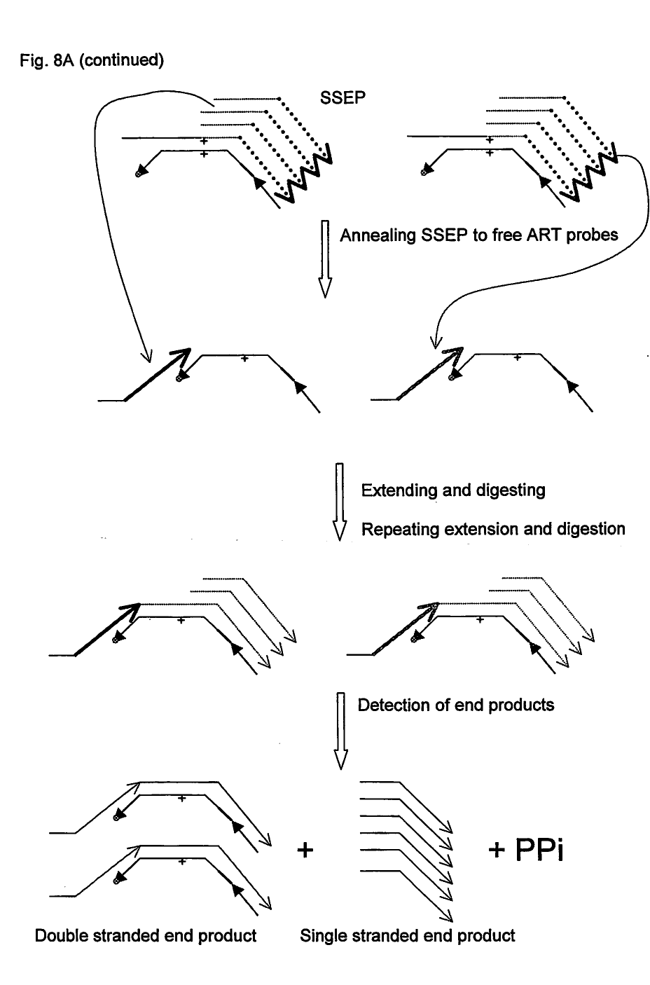

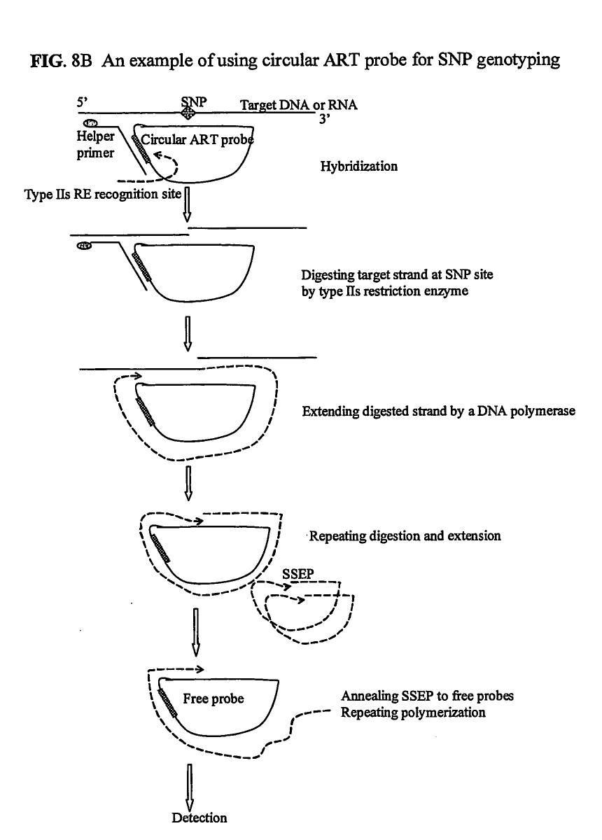

Another example of a method of the invention is outlined below (FIG. 8).

A CELA reaction using linear probes is shovro in FIG. 8A and CELA reaction using circular probes is shown in FIG. 8B. The ART probes comprise a type IIS restriction site with its recognition site on the 3′ side of the target complementary portion (FIG. 8A) or the 5′ side of the target complementary portion (FIG. 8B) and its cleavage site on the target complementary portion. The cleavage site of the type IIS restriction enzyme corresponds to 3′ SNP nucleotide of the target strand, whereas the cleavage site on the probe is modified and is resistant to cleavage. The ART probes on the left hand side comprise target complementary portion matching to the sequence of allele 1; the ART probes on the right hand side comprise target complementary portion matching to the sequence of allele2 (FIG. 8A). A target RNA or DNA sequence is allele-specifically hybridized to target complementary portions of ART probes, while helper-primer anneal to both ART probes and a target sequence. The target region hybridized to the helper primer is adjacent or substantially adjacent to the target region hybridized to the ART probe. The 3′ end sequence of the helper primer (FIG. 8A) that hybridizes to a sequence 3′ to the type IIS restriction site is short, may comprise 2 to 15 nucleotides, preferably comprise 3 to 10 nucleotides, or more preferably comprise 4 to 8 nucleotides. In FIG. 8A, the 3′ end of helper-primer is extended by a DNA polymerase using ART probe as template and a double stranded functional type IIS restriction recognition site is created. In FIG. 8B, the double stranded functional type IIS restriction recognition sites are created by hybridization between helper primers and ART probes. The target strands on the double stranded target complementary portions of ART probes are digested by a type IIS restriction enzyme (for example Fok I), while ART probe strands are resistant to cleavage due to modified nucleotides. The 3′ ends of the digested strands are extended by a DNA polymerase using the ART probes as templates. The digestion and extension are repeated multiple times to generate multiple copies of single stranded end products (SSEP). In FIG. 8A, the probes are linear molecules, the SSEP are complementary to the sequence 5′ to the restriction digestion site of the probe that includes a 5′ template portion sequence and part of the target complementary portion sequence of the probe. Because the 3′ template portion and 5′ template portion of the probe comprise identical or nearly identical sequences, the 3′ end of SSEP is complementary to 3′ template portion. The SSEP anneal to 3′ template portions of free probes and are extended by the DNA polymerase, and double stranded ART probes are formed that trigger repeated digestion and extension. In FIG. 8B, the probes are circular molecules, and the SSEP are complementary to the whole sequence of the ART probe. The SSEP anneal fully or partially to free ART probes and are extended by the DNA polymerase, therefore double stranded ART probes are formed that trigger repeated digestion and extension. It is preferred that extension of the 3′ ends of the SSEP or the 3′ ends of the digested strands may be carried by the DNA polymerase having strand displacement activity or containing other strand displacement factors. Because the allele-specific ART probes comprise different template portions, the SSEP produced by the allele-specific ART probes are different and can be distinguished by a detection method. A preferred detection method is the use of SSEP DNA enzyme that is described in the next example.

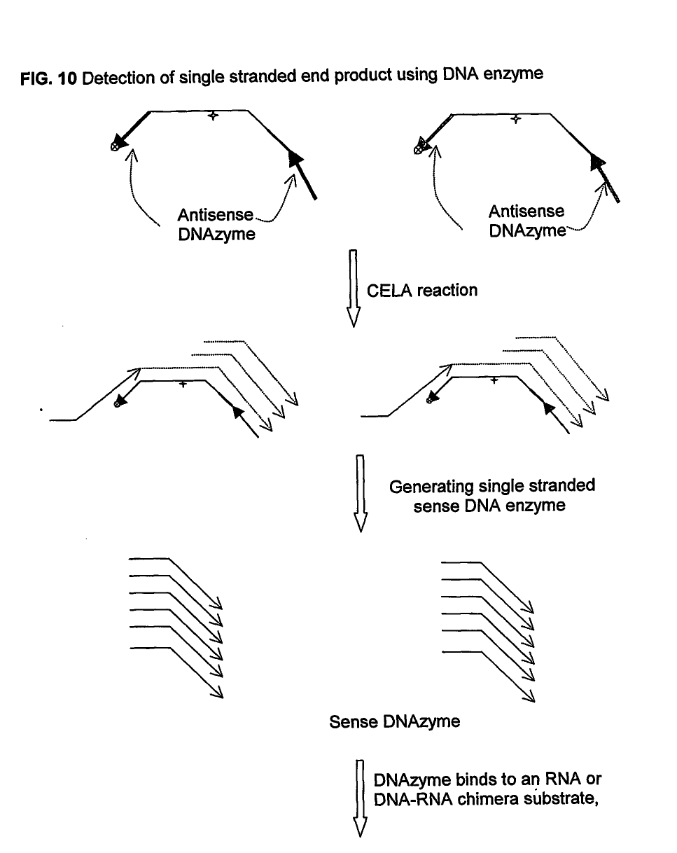

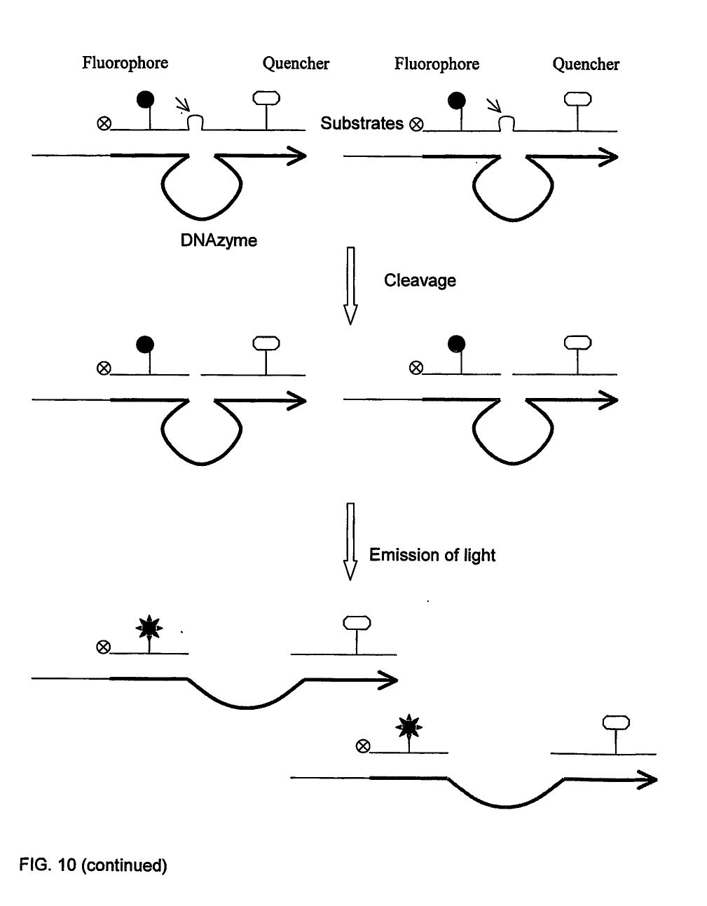

An example of a method of the invention for DNA enzyme mediated detection of single stranded end products—SSEP is outlined below (FIG. 10):

The template portion of ART probe comprises a complementary (antisense) sequence of a DNAzyme, for example 10-23 DNAzyme. During CELA reaction, SSEP are produced that contain active (sense) copies of DNAzymes. The DNA enzyme binds an RNA or DNA-RNA chimeric reporter substrate which contains fluorescence resonance energy transfer fluorophores incorporated on either side of a DNAzyme cleavage site. Cleavage of this reporter substrate produces an increase in fluorescence that is indicative of successful amplification of the SSEP.

I. Material

A. Target Sequence

The target sequence, which is the object of hybridization to ART probe and helper primer, and initiation of amplification and detection, can be any nucleic acid. The target sequence can be any RNA, cDNA, genomic DNA, disease-causing microorganism DNA, any virus DNA, RNA etc. The target sequence may also be DNA, RNA treated by chemical reagents, various enzymes and physical exposure.

B. Amplification Repeat Template (ART) Probe

Amplification Repeat Template (ART) probes are single-stranded or partially double stranded linear or circular nucleic acid molecules, generally containing between 20 to 2000 nucleotides, preferably between about 30 to 300 nucleotides, and most preferably between about 40 to 150 nucleotides. Portions of ART probe have specific functions making the ART useful for combined exponential and linear amplification (CELA). An ART probe comprises the target complementary portions, template portions, enzyme acting portions, with or without a 3′ end block portion. The ART probe may comprise a helper primer that makes some part of probe double stranded. The ART probe may comprise antisense DNA enzyme or antisense RNA enzyme. An ART probe may not comprise all portions and may comprise additional portions.

1. Target Complementary Portion

The target complementary portion of probe can be any length that supports specific and stable hybridization between the target complementary portion and the target sequence. For this purpose, a length of 9 to 90 nucleotides for target complementary portion is preferred, and 15 to 40 nucleotides long is most preferred.

The target complementary portion of the probe is complementary or substantially complementary to a target region of interest. The target region of interest chosen may be any desirable sequence, which may comprise SNP site, mutation sequence, methylation site, splicing site, restriction site, and any particular sequence of interest.

The target complementary portion of the probe becomes double stranded after specific hybridization between the target and the probe. The target complementary portion of the probe hybridizes to the target region of interest, whereby one or more than one or part of the enzyme acting portion(s) of said probe is partially or fully functional. In some embodiments, the target region that hybridizes to the target complementary portion of the probe is digested or nicked by digesting agents that act on the enzyme acting portions of the probe. In another embodiment, the target complementary portions of the probes hybridize to free 3′ end(s) of the target sequence(s), which are extended by a DNA polymerase using said probes as templates, whereby other enzyme acting portions on the probes become double stranded and functional. 2. Enzyme acting portions An ART probe comprises at least one enzyme acting portion. An enzyme acting portion generally has the following properties: (a) it is usually non-functional when single stranded, and becomes fully or partially functional when fully double stranded or partially double stranded; (b) it either supports digestion of one strand of a nucleic acid duplex for example RNase H digestion sites, supports repeated digestion and extension for example restriction enzyme site, or supports repeated polymerization for example RNA polymerase promoter. An ART probe may usually require comprising at least one enzyme acting portion that supports repeated digestion and extension, or require comprising one enzyme acting portion that supports digestion and another enzyme acting portion that supports repeated polymerization. The enzyme acting portion may comprise, but is not limited to, a restriction enzyme site, RNase H digestion site, other nuclease digestion site, and an RNA polymerase promoter sequence. The enzyme acting portions may comprise the combination of the RNase H acting sequences and the RNA polymerase promoter or the combination of the RNase H acting sequences and the nuclease digestion sites or the combination of the nuclease digestion sites and the RNA polymerase promoter or the combination of more than one of the nuclease digestion sites. The enzyme acting portions may comprise any other combination of the sequences for the mentioned enzymes or other enzymes having similar activities.

The enzyme acting portion may be located in any place of the probe, for example within the target complementary portion, or on either side of the target complementary portion, or on template portions.

If the enzyme acting portion comprises nuclease digestion sites, the nucleotides on the nuclease digestion sites of probe may be modified so that the probe is resistant to nuclease digestion whereas the opposite strand of the probe is sensitive to digestion. Any means for modifying nucleotide that make nucleotides resistant to nuclease cleavage can be used, for example phosphorothioate linkages between nucleotides, methylated nucleotides. The phosphorothioated nucleotides are preferred. Alternatively, if the enzyme acting portion comprises a nicking restriction enzyme site, for example N.Bpu10I site, N.BstSE site, the nucleotide modification on the probe sequence is not required.

In some embodiments, the enzyme acting portion comprises restriction site(s) (FIG. 1 and 2). The restriction site has a restriction enzyme recognition sequences and a cleavage site. Restriction cleavage site on ART probes may comprise modified nucleotides so that the probe is resistant to nuclease digestion. The ART probe may comprise one or more than one restriction sites which may be located within the target complementary portion or on either side of the target complementary portion.

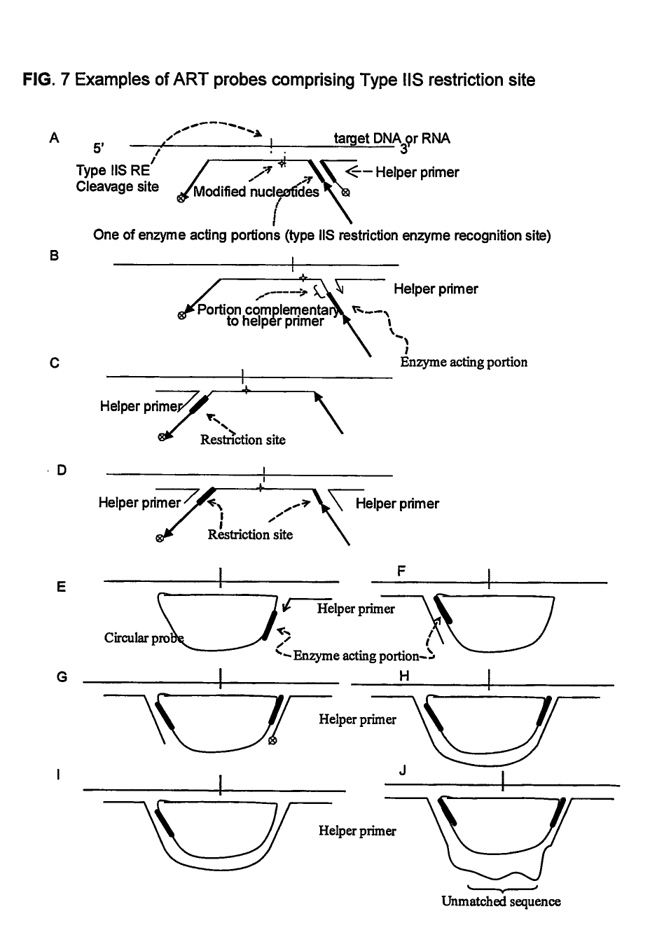

In some embodiments, the ART probe comprises type IIS restriction enzyme site as one of enzyme acting portions (FIG. 1D, IE, 1I, 1K, 1L and FIG. 7). Because the type IIS enzymes cut several bases away from restriction recognition site, the cleavage site can be or is preferred to be located on the target complementary portion of the probe. The nucleotide(s) on the cleavage site of the target complementary portion of the probe is modified to block cleavage of the probe. For SNP genotyping, it is preferred that the digestion site on target molecule is also the SNP or mutation site, and preferably the type IIS restriction enzyme cleaves at 3′ of the SNP nucleotide or mutation nucleotide. For detection of nucleotide methylation, it is preferred that the digestion site on target molecule is also the methylation site.

To be functional, the type IIS restriction site of the probe must be converted to double stranded form for both its recognition and cleavage sites. In the beginning of a CELA reaction, the target initiates the amplification through specific hybridization to the probe. This hybridization creates double stranded cleavage site for type IIS restriction enzyme.

In the same stage of the reaction, the type IIS restriction recognition site becomes double stranded through the following ways. First, the type IIS restriction recognition sequence hybridizes to a helper primer (FIG. 1D, 1E, 1J, 1K, 1L, FIG. 7A, 7C, 7D, 7F, 7G, 7H, 7I, 7J) therefore become double stranded. Second, the target complementary sequence of a helper primer hybridizes to a target region that is adjacent to the hybridization region of the ART probe (FIG. 7B, 7E), while the 3′ end sequence of the helper primer anneals to a sequence 3′ to the type IIS restriction recognition site. The 3′ end of helper primer is extended by a DNA polymerase using the probe as template; therefore a functional double stranded type IIS restriction recognition site is formed.

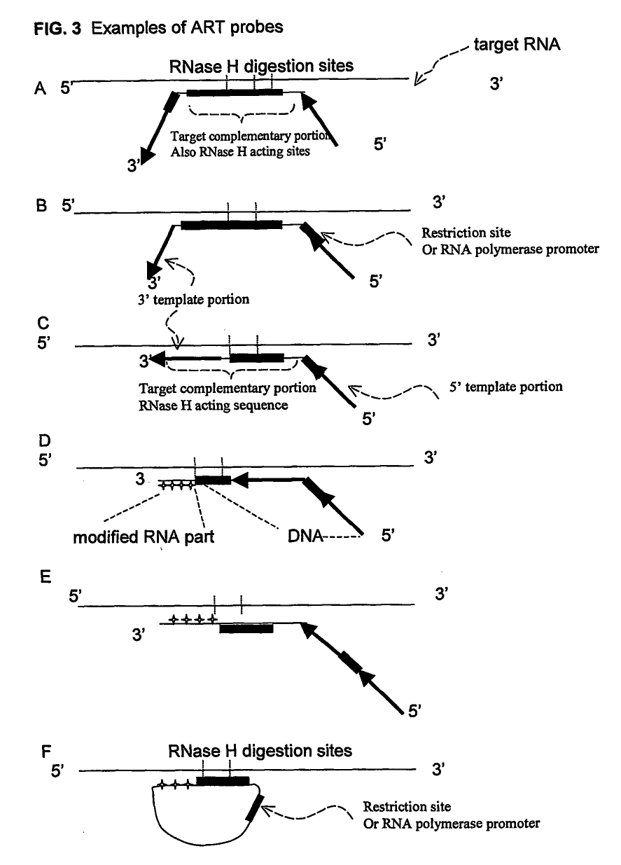

In some embodiments, when the target sequence is RNA, in the beginning of a CELA reaction, the target RNA initiates the amplification through specific hybridization to the probe (FIG. 3, 4). This hybridization creates a double stranded functional enzyme acting portion sequence for a double strand specific ribonuclease. For example, the target RNA sequence on RNA/DNA duplex may be digested by RNase H at various non-specific sites. In one embodiment, a part of the target complementary portion (preferable the 3′ part sequence) may be made by RNA (FIG. 3D, 3E, 3F). The hybridization between target RNA sequence and the target complementary portion of ART probe forms a part with RNA/DNA hybrid and a part with RNA/RNA hybrid. The target RNA on the RNA/RNA hybrid is resistant to RNase H cleavage therefore the target RNA is not completely digested away with RNase H. This approach leaves a part of RNA sequence intact, so that the 3′ end of the digested RNA can be extended by a DNA polymerase. It is also preferred that the RNA part on ART probes is modified so that it is not digested by any nuclease. The modified nucleotides may comprise phosphorothioate linkages.

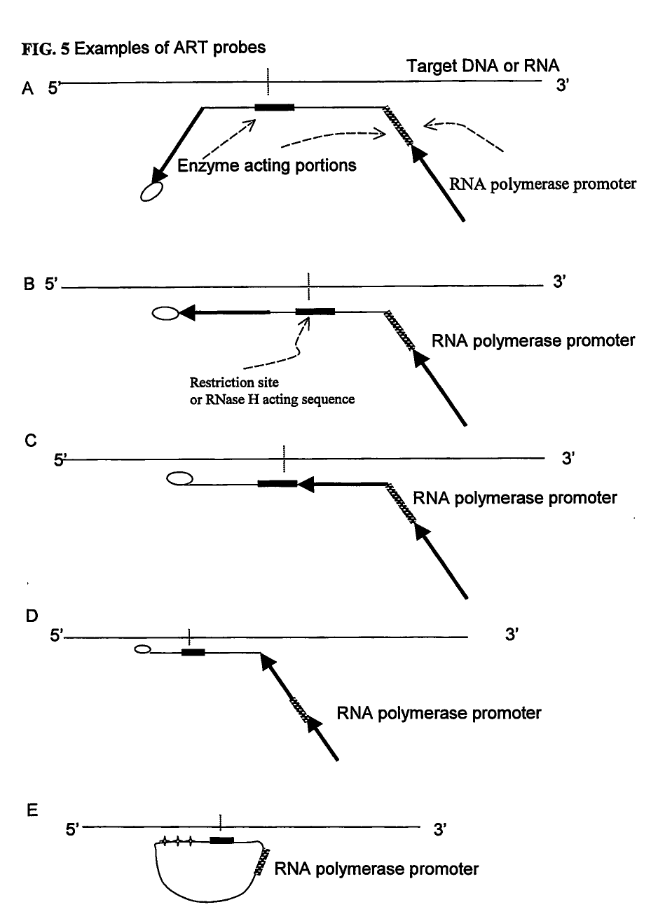

In some embodiments, one of enzyme acting portions is a RNA polymerase promoter sequence (FIG. 5, 6). RNA polymerase promoter comprises the sequence of a promoter recognized by an RNA polymerase and a transcription initiation region which is located between the template portion and the sequence of the promoter. The promoter may be the promoter for any suitable RNA polymerase. Examples of RNA polymerase are polymerases from E.coli and bacteriophages T7, T3 and SP6. Preferably the RNA polymerase is a bacteriophage-derived RNA polymerase, in particular the T7 polymerase. Because promoter sequences are generally recognized by specific RNA polymerases, the cognate polymerase for the promoter portion of the ART probe should be used for transcriptional amplification. The promoter sequence can be located anywhere in the probe. If the probe is linear, it is preferred that the promoter is immediately adjacent to the target complementary portion and is oriented to promote transcription toward the 5′ end of the ART probe.

3. Template Portions

The ART probe comprises at least one template portion. If the ART probe is linear molecule, two template portions comprising identical or nearly identical sequences are preferred. If the ART probe is circular molecule, the entire ART probe severs as template, so the ART probe may be regarded as comprising one template with other functional portions embedded in it. The template portions may have any desired length. The template portions serve as templates for generating multiple copies of SSEP and as templates for SSEP annealing. For this purpose, a length of 6 to 300 nucleotides for the template portion is preferred, with template portions 15 to 150 nucleotides long being most preferred. The template portions can have any desired sequence. In general, the sequence of the template portions can be chosen such that it is nether significantly similar to any sequence in the nucleic acid sample, nor to any sequence of other ART probes in the CELA reaction.

In some embodiments, the template portions may overlap the target complementary portion (FIG. 1F, FIG. 3C, 3D, FIG. 5B, 5C). In general, for linear probes between two template portions there is at lease one enzyme acting portion, which may comprise RNA polymerase promoter or restriction site. The template portion in the 3′ region of the ART probe is referred to as the 3′ template portion; the template portion in the 5′ end of the ART probe is referred to as the 5′ template portion. The 5′ template portion is usually located at the most 5′ end of the ART probe. The 3′ template portion may be located anywhere for example on either side of target complementary portion (FIG. 1B, 1C, 1D, 1E, 1G), or within target complementary portion (FIG. 1F).

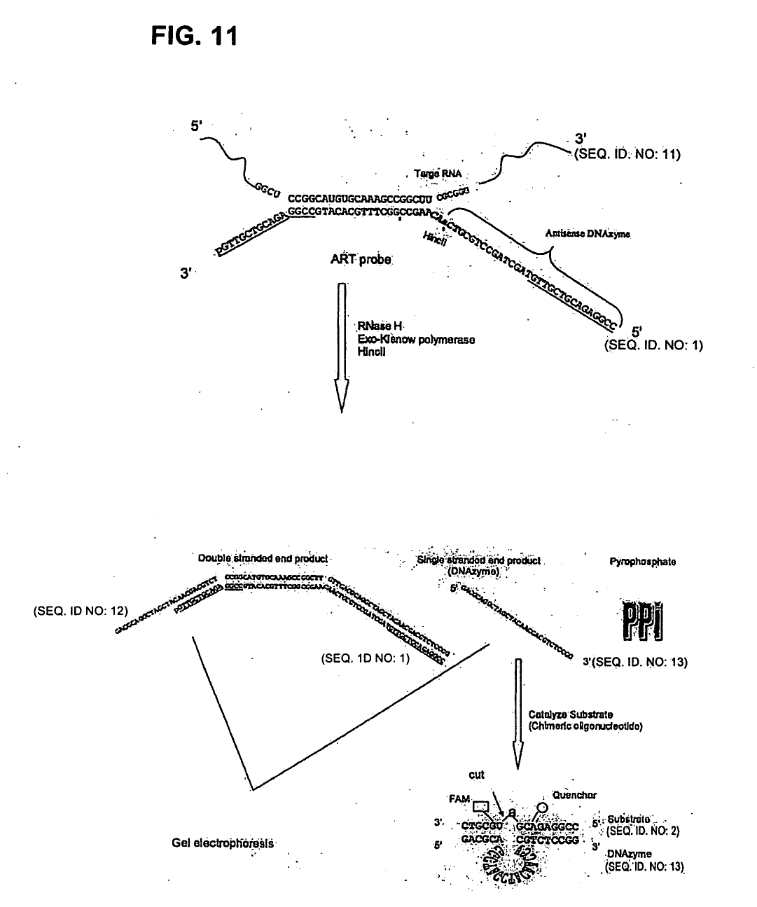

In some embodiments (FIG. 10, FIG. 11), when DNA enzyme is used for detecting single stranded end products (SSEP), the ART probe comprises a catalytically inactive antisense DNA enzyme sequence that is complementary to an active DNA enzyme, for example the 10-23 DNAzyme, 8-17 DNAzyme or other DNAzyme. If the probe is linear molecule, the antisense DNA enzyme sequence is preferably located in the 5′ template portion or the template portion with or without surrounding portion sequences. If the probe is circular molecule, the antisense DNA enzyme sequence may be present in any place of the probe. In some embodiments, when the SSEP are RNA molecules which are designed to be RNA enzyme, the ART probe may comprise antisense RNA enzyme in the template portion of the probes.

4. The 3′ End Block Portion

It is preferred that the ART probe is blocked by a blocking group at its 3′ end, or is circular molecule (FIG. 1H, , 1I, 1J, 1K, 1L, FIG. 3F, FIG. 5E, FIG. 7E, 7F, 7G, 7H, 7I, 7J) such that it is not extendible by a polymerase. The blockage of the 3′ end of the ART probe can be achieved by any means known in the art. Blocking groups are chemical moieties which can be added to a nucleic acid to inhibit nucleic acid polymerization catalyzed by a nucleic acid polymerase. Blocking groups are typically located at the terminal 3′ end of an ART which is made up of nucleotides or derivatives thereof. By attaching a blocking group to a terminal 3′ OH, the 3′ OH group is no longer available to accept a nucleoside triphosphate in a polymerization reaction.

Numerous different groups can be added to block the 3′ end of a probe sequence. Examples of such groups include phosphate group, alkyl groups, non-nucleotide linkers, phosphorothioate, alkane-diol residues, peptide nucleic acid, and nucleotide derivatives lacking a 3′ OH (e.g., cordycepin).

The 3′ end of the ART probe may also be attached to a solid support, such as glass slides, nylon membrane, plastic material so that CELA reaction can performed on the solid support.

5. Other Moieties of ART Probe

In certain embodiments, ART probe may comprise one or more moieties incorporated into 5′ or 3′ terminus or internally of primers that allow for the affinity separation of products associated with the label from unassociated part. Preferred capture moieties are those that can interact specifically with a cognate ligand. For example, capture moiety can include biotin, digoxigenin etc. Other examples of capture groups include ligands, receptors, antibodies, haptens, enzymes, chemical groups recognizable by antibodies or aptamers. The capture moieties can be immobilized on any desired substrate. Examples of desired substrates include, e.g., particles, beads, magnetic beads, optically trapped beads, microtiterplates, glass slides, papers, test strips, gels, other matrices, nitrocellulose, nylon. For example, when the capture moiety is biotin, the substrate can include streptavidin.

In some embodiments, the ART probes or a set of ART probes are attached on a solid support, preferably the 3′ end of ART probes are attached on a solid support. The solid support can include any solid material to which oligonucleotides can be coupled. This includes materials such as acrylamide, cellulose, nitrocellulose, glass, polystyrene, polyethylene vinyl acetate, polypropylene, polymethacrylate, polyetbylene, polyethylene oxide, polysilicates, polycarbonates, teflon, fluorocarbons, nylon, silicon rubber, polyanhydrides, polyglycolic acid, polylactic acid, polyorthoester, polypropylfirmerate, collagen, glycosaminoglycans, and polyamino acids. Solid-state substrates can have any useful form including thin films or membranes, beads, bottles, dishes, fibers, woven fibers, shaped polymers, particles and microparticles. A preferred form for a solid-support is glass slides.

The ART probes immobilized on a solid-state substrate allow capture of specific target molecules and amplification on a solid-state detector. Such capture provides a convenient means for gene expression profiling and detecting multiple targets. For example, 3′ ends of ART probes specific for multiple different target sequences can be immobilized on a glass slide, each in a different spot. Amplification of end products specific for target sequences will occur only on those spots corresponding to ART probes for which the corresponding target sequences were present in a sample.

Methods for immobilization of oligonucleotides to solid-state substrates are well established. ART probes can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A preferred method of attaching oligonucleotides to solid-state substrates is described by Guo et al., Nucleic Acids Res. 22:5456-5465 (1994).

6. Helper Primer

The ART probe may comprise helper primer(s), which comprises at least one portion complementary or substantially complementary to a part of said probe (FIG. 1D, 1E, 1J, 1K, 1L, FIG. 7). The helper primer may comprise a 3′ end blocking moiety, whereby the 3′ end of said helper primer is not extendible by a DNA polymerase (FIG. 1J, 1K, 1L, 7A, 7G). The helper primer may not comprise a 3′ end blocking moiety, whereby the 3′ end of said helper primer is extendible by a DNA polymerase (FIG. 7B).

The helper primer may comprise sequence complementary to enzyme acting portion(s) with or without flanking sequences or to part of enzyme acting portion(s) of the probe, whereby the hybridization between said helper primer and said probe makes the enzyme acting portion(s) double stranded or partially double stranded (FIG. 1D, 1E, 1J, 1K, 1L, 7A, 7C, 7D, 7F, 7G, 7H, 7I, 7J). The sequence on the helper primer complementary to enzyme acting portion(s) or part of enzyme acting portion(s) of the probe can be any length that supports hybridization between the probe and the helper primer and makes the enzyme acting portion(s) functional or partially functional.

The helper primer may comprise 3′ end sequence which is extendable and is complementary to a sequence 3′ to one of the enzyme acting portions of the probe. Hybridization between the helper primer and the probe, and extension of 3′ end of said helper primer by a DNA polymerase make the enzyme acting portion double stranded and fully functional or partially functional (FIG. 7B, 7E). The 3′ end sequence of the helper primer that is complementary to a sequence 3′ of one of the enzyme acting portions of the probe has a length of 2 to 15 nucleotides, or preferably 3 to 10 nucleotides, or even preferably 4 to 8 nucleotides.

The helper primer may further comprise at least one target complementary portion, which hybridizes to a target region that is adjacent or substantially adjacent to the target region complementary to said probe (FIG. 1J, 1K, 7A-G). The target complementary portion of the helper primer can be any length that supports specific and stable hybridization between the helper primer and the target sequence. For this purpose, a length of 9 to 60 nucleotides for the target complementary portion of the helper primer is preferred, and 15 to 40 nucleotides long is most preferred.

The helper primer may comprise both 3′ and 5′ target complementary portions. The target region complementary to the ART probe is located in the middle of the target regions complementary to the helper primer and is adjacent or substantially adjacent to the target regions complementary to the helper primer (FIG. 7H-J).

The helper primer may comprise other sequences that are complementary to any portions of the ART probe (FIG. 1K, 1L). The helper primer may comprise other sequences that may not be complementary to any portions of the ART probe (FIG. 7J).

Helper primer may have any length, as long as it efficiently hybridizes to the ART probe and/or the target sequence. Helper primer may comprise any desired nucleotide modifications.

C. Detection Labels

To aid in detection and quantitation of nucleic acids amplified using CELA, detection labels can be directly incorporated into amplified nucleic acids or can be coupled to detection molecules. As used herein, a detection label is any molecule that can be associated with amplified nucleic acid, directly or indirectly, and which results in a measurable, detectable signal, either directly or indirectly. Many such labels for incorporation into nucleic acids or coupling to nucleic acid or antibody probes are known to those of skill in the art. Examples of detection labels suitable for use in CELA are radioactive isotopes, fluorescent molecules, phosphorescent molecules, enzymes, antibodies, and ligands.

Examples of suitable fluorescent labels include but are not limited to fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, Oreg. and Research Organics, Cleveland, Ohio.

Labeled nucleotides are a preferred form of detection label since they can be directly incorporated into the products of CELA during synthesis. Examples of detection labels that can be incorporated into amplified DNA or RNA include nucleotide analogs such as BrdUrd (Hoy and Schimke, Mutation Research 290:217-230 (1993)) BrUTP (Wansick et al, J. Cell Biology 122:283-293 (1993))and nucleotides modified with biotin (Langer et al, Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (BUDR triphosphate, Sigma), and a preferred nucleotide analog detection label for RNA is Biotin-16-uridine-5′-triphosphate (Biotin-16-dUTP, Boebringher Mannheim). Fluorescein, Cy3, and Cy5 can be linked to dUTP for direct labeling. Cy3.5 and Cy7 are available as avidin or anti-digoxygenin conjugates for secondary detection of biotin- or digoxygenin labeled probes.

Detection labels that are incorporated into amplified nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4methoxyspiro[1,2,-dioxetane-3-2′-(5′-chloro)tricyclo [3.3.1.1.sup.3,7 J ]decane]-4-yl) phenyl phosphate;Tropix, Inc.).

A preferred detection label for use in detection of amplified RNA is acridinium-ester-labeled DNA probe (GenProbe, Inc., as described by Arnold et al., Clinical Chemistry 35:1588-1594 (1989)). An acridinium-ester-labeled detection probe permits the detection of amplified RNA without washing because unhybridized probe can be destroyed with alkali (Arnold et al. (1989)).

Molecules that combine two or more of these detection labels are also considered detection labels. Any of the known detection labels can be used with the disclosed probes, tags, and method to label and detect nucleic acid amplified using the disclosed method. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. Such methods can be used directly in the disclosed method of amplification and detection. As used herein, detection molecules are molecules which interact with amplified nucleic acid and to which one or more detection labels are coupled.

D. Reporter Substrate

A reporter substrate molecule can be an RNA or DNA-RNA chimera which contain fluorescence resonance energy transfer fluorophores incorporated on either side of a DNAzyme cleavage site (Santoro et al. Biochemstry 1998, 37, 13330-13342). Any fluorophores and any quenchers can be incorporated at any desired places into a reporter substrate. One example is that the reporter 6-carboxytluorescein (RAM) is incorporated at the 5′ end, and the quencher 6-carboxytetramethylrhodamine (TAMRA) or 4-(4-dimethylaminophenylazo)benzoic acid (DABCYL) is incorporated internally. Any blocking moiety such as 3′ phosphate group can be added to the 3′ end to prevent extension by DNA polymerase during reaction. Cleavage of this reporter substrate produces an increase in fluorescence that is indicative of successful amplification.

E. DNA Polymerases

For combined exponential and linear amplification (CELA), it is preferred that a DNA polymerase is capable of displacing the strand complementary to the template strand, termed strand displacement, and lack a 5′ to 3′ exonuclease activity. Strand displacement may be necessary to result in synthesis of multiple copies of the SSEP with long sequence which may be more than 100 nucleotides, or more than 50 nucleotides. However, for producing short SSEP which may be in a range of 3 to 50 nucleotides, the strand displacement may by not necessary. A 5′ to 3′ exonuclease activity, if present, might result in the destruction of the synthesized strand. It is also preferred that DNA polymerases for use in the disclosed method are highly processive. Preferred DNA polymerases are bacteriophage .phi.29 DNA polymerase (U.S.Pat. Nos. 5,198,543 and 5,001,050 to Blanco et al.), phage M2 DNA polymerase (Matsumoto et al., Gene 84:247 (1989)), phage .phi.PRDI DNA polymerase (Jung et al., Proc. Natl. Acad: Sci. USA 84:8287 (1987)), VENT.RTM. DNA polymerase (Kong et al., J. Biol. Chem. 268:1965-1975 (1993)), Klenow fragment of DNA polymerase I (Jacobsen et al., Eur. J Biochem. 45:623-627 (1974)), T5 DNA polymerase (Chatteijee et al., Gene 97:13-19 (1991)), PRD1 DNA polymerase (Zhu and Ito, Biochim. Biophys. Acta. 1219:267-276 (1994), modified T7 DNA polymerase (Tabor and Richardson, J. Biol. Chem. 262:15330-15333 (1987); Tabor and Richardson, J. Biol. Chem. 264:6447-6458 (1989); Sequenase.TM. (U.S. Biochemicals)), T4 DNA polymerase holoenzyme (Kaboord and Benkovic, Curro Biol. 5:149-157 (1995), Bca polymerase (Takara) and Bst polymerase (NEB). .phi.29 and Bst DNA polymerases are most preferred.

Strand displacement can be facilitated through the use of a strand displacement factor, such as helicase. It is considered that any DNA polymerase that can perform strand displacement in the presence of a strand displacement factor is suitable for use in the disclosed method, even if the DNA polymerase does not perform strand displacement in the absence of such a factor. Strand displacement factors useful in CELA include, but is not limited to, BMRF 1 polymerase accessory subunit (Tsurumi et al., J. Virology 67(12):7648-7653. (1993)), adenovirus DNA-binding protein (Zijderveld and van der Vliet, J. Virology 68(2): 1158-1164 (1994)), herpes simplex viral protein ICP8 (Boehmer and Lehman: J. Virology 67(2):711-715 (1993); Skaliter and Lehman, Proc. Natl. Acad. Sci. USA 91(22):10665-10669 (1994)), single-stranded DNA binding proteins (SSB; Rigler and Romano, J. Biol. Chem. 270:8910-8919 (1995)), and calf thymus helicase (Siegel et al., J. Biol. Chem. 267:13629-13635 (1992)).

E. RNA Polymerases

Any RNA polymerase which can carry out transcription in vitro and for which promoter sequences have been identified can be used in the disclosed CELA method. Stable RNA polymerases without complex requirements are preferred. Most preferred are T7 RNA polymerase (Davanloo et al., Proc. Natl. Acad. Sci USA 81:2035-2039 (1984)) and SP6 RNA polymerase (Butler and Chamberlin, J. Biol. Chem. 257:5772-5778 (1982)) which are highly specific for particular promoter sequences (Schenbom and Meirendorf, Nucleic Acids Research 13:6223-6236 (1985)). Other RNA polymerases with this characteristic are also preferred. Because promoter sequences are generally recognized by specific RNA polymerases, the ART probe should contain a promoter sequence recognized by the RNA polymerase that is used. Numerous promoter sequences are known and any suitable RNA polymerase having an identified promoter sequence can be used.

F. Restriction Enzymes and Ribonuclease

The disclosed method may use restriction enzymes (also referred to as restriction endonucleases) for cleaving one strand of double stranded nucleic acids. Other nucleic acid cleaving agents may also be used. Preferred nucleic acid cleaving agents are those that cleave nucleic acid molecules in a sequence-specific manner. Many restriction enzymes are known and can be used with the disclosed method. Restriction enzymes generally have a recognition sequence and a cleavage site.

In some embodiments, digestion of target RNA hybridized to the probe is carried out with a ribonuclease. Such ribonucleases digest RNA strand found on double-stranded RNA/DNA hybrid. An example of such ribonuclease useful in the practice of this invention is RNase H. RNase H is a RNA specific digestion enzyme, which cleaves RNA found in DNA/RNA hybrids in a non-sequence-specific manner. Other ribonucleases and enzymes may be suitable to nick or partially digest RNA from RNA/DNA strands, such as Exo III and reverse transcriptase.

The materials described above can be packaged together in any suitable combination as a kit useful for performing the disclosed method.

II. Method

The present invention provides specially designed probes that allow for combined exponential and linear amplification. The probes are either linear molecules or circular molecules.

The initiation of the combined exponential and linear amplification (CELA) depends on the specific hybridization between target sequences and ART probes including helper primers. In the presence of a DNA or RNA molecules having the target sequence, the target complementary portions of ART probes hybridize to the target sequences and become double stranded, which form functional or partial functional enzyme acting portion sequences and allow for the target sequence to be extended by a DNA polymerase or be digested or partially digested by a digesting agent then 3′ ends of digested strands are extended by a DNA polymerase, therefore create other functional enzyme acting portions. The subsequent repeated polymerization generates multiple copies of single stranded end products (SSEP) which then anneal to free ART probes, prime new extension, and generate new SSEP.

Linear amplification takes place when individual ART probes produce multiple copies of SSEP. Exponential amplification takes place during repeated replication of SSEP that anneal to free ART probes and prime new SSEP generation. When there are free ART probes in a reaction, the reaction may combine exponential and linear amplifications; while after all ART molecules hybridize to SSEP and become double stranded the linear amplification is dominant.

Following nucleic acid amplification in the disclosed methods, the amplified double stranded end product, single stranded end product (SSEP) and pyrophosphate (PPI) can be detected and quantified using any of the conventional detection systems such as detection of fluorescent labels, enzyme-linked detection systems, microarray hybridization, capillary and gel electrophoresis, fluorescence polarization, mass spectrometry, Fluorescence Resonance Energy Transfer (FRET), Time-resolved fluorescence detection, electrical detection, and luminescence detection.

The invention also provides a detection method using DNAzyme. The ART probe comprises a complementary (antisense) sequence of a DNAzyme. During amplification, single-stranded end products are produced that contain active (sense) copies of DNAzymes that cleave a reporter substrate included in the reaction mixture.

The major steps of CELA reaction are described as follows. All steps may be performed in a single tube as a single reaction, or performed in different tubes as separated reactions. The single reaction format is preferred.

A. Target Specific Hybridization

ART probes or a set of ART probes or a mixture of ART probes and helper primer are incubated with a sample containing target DNA, RNA, or both, under suitable hybridization conditions, so that double stranded DNA/DNA or RNA/DNA hybrids in the target complementary portions of ART probe or helper primers are formed, whereby one of the enzyme acting portions of probes is partially or fully functional. If one of the enzyme acting portions is restriction site which is located within the target complementary portion, the hybridization makes the restriction site double stranded therefore functional. If one of the enzyme acting sequence is type IIS restriction site of which the cleavage site is located within the target complementary portion, the hybridization makes the restriction cleavage site double stranded therefore the type IIS restriction site is partially functional. If one of the enzyme acting sequence is RNase H acting sequence which overlap the target complementary portion, the hybridization makes the RNase H acting sequence double stranded therefore fully functional. A stringent hybridization condition allows subsequent amplification to be dependent on the perfect match between a target sequence and ART probe so that allele discrimination can be achieved.

The helper primer, if used in CELA reaction, may hybridize to both target and ART probe that therefore facilitate specific hybridization between the target and the probe.

B. Causing All Enzyme Acting Portions of Probes Double Stranded and Fully Functional

In some embodiments, when target nucleic acid is RNA, in the step (A) a functional enzyme acting portion, the RNase H digesting sites, is formed (FIG. 3, 4). To cause all other enzyme acting portions of probes double stranded and fully functional, this step comprises: digesting RNA strand by RNase H and extending 3′ end of digested strand using the probe as template by a DNA polymerase, whereby all other enzyme acting portions on the probes become double stranded and functional. The other enzyme acting portions on the probes may comprise restriction site or RNA polymerase promoter or both restriction site and RNA polymerase promoter. In further embodiments, extending the 3′ end of partially digested strand may further comprise strand displacing by the DNA polymerase or other strand displacement factors.

RNase H is a RNA specific digestion enzyme which cleaves RNA found in DNA/RNA hybrids in a non-sequence-specific manner. To prevent complete digestion away of RNA strand, a portion of target complementary portion of ART is made by RNA (FIG. 3D), thus RNA/RNA hybrid is resistant to the digestion by RNase H.

In some embodiments, when one of enzyme acting portions is restriction site and is located within the target complementary portion of ART probe, the step (A) causes said restriction site fully functional. To cause all other enzyme acting portions of probes double stranded and fully functional, this step comprises digesting opposite strand of said probes and extending 3′ end of digested strand using the probe as template by a DNA polymerase, whereby all other enzyme acting portions on probes become double stranded and functional. The other enzyme acting portions on said probes may comprise other restriction site, RNA polymerase promoter or both restriction site and RNA polymerase promoter. Alternatively, said restriction site is the only enzyme acting portion on the probe. In further embodiments, extending the 3′ end of the digested strand may fulther comprise strand displacing by the DNA polymerase or other strand displacement factors.

In some embodiments, when one of the enzyme acting portions is type IIS restriction site with the cleavage site of the type IIS restriction site on target complementary portion of the probe and the recognition site of the type IIS restriction site on either side of target complementary portion of the probe, and step (a) causes the target complementary portions of the probe double stranded, whereby a functional cleavage site of the type IIS restriction site is formed, the step (b) comprises: annealing helper primers to the probes and causing the recognition sequence of the type IIS restriction site double stranded. In one embodiment, annealing helper primers to the probes and causing the recognition sequence of the type IIS restriction site double stranded comprises: annealing the helper primers directly to the type IIS restriction enzyme recognition sequence with or without flanking sequences whereby double stranded recognition sequence of the type IIS restriction site is formed. In another embodiment, annealing helper primers to the probes and causing the recognition sequence of the type IIS restriction site double stranded comprises: annealing the 3′ end sequence of the helper primer to a sequence 3′ of the type IIS restriction recognition sequence and extending the 3′ end sequence of the helper primer by a DNA polymerase using the probe as template, whereby double stranded recognition sequence of the type IIS restriction site is formed.

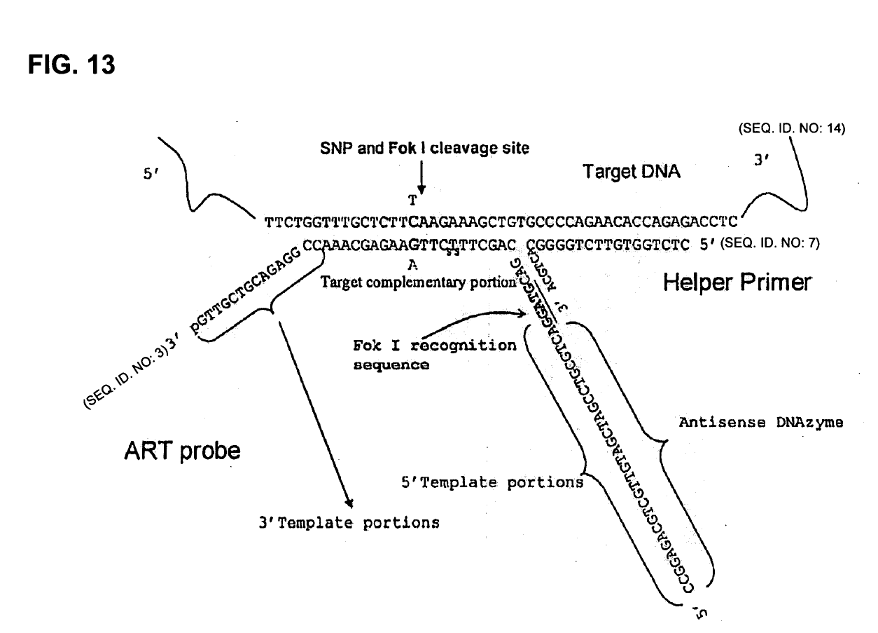

For genotyping, especially for genotyping SNPs or nucleotide methylation, the target specific ART probes, helper primer and type IIS restriction enzyme are included in the reaction (FIG. 8 and FIG. 13). The advantage of using type IIS restriction enzyme is that the target sequence for analyzing does not need to be restricted to contain any specific sequence, for example a restriction enzyme site. An universal restriction enzyme can be used in all detection reactions. The type IIS restriction recognition site is usually located on either side of target complementary portion of ART probe (FIG. 1D and 1E, 7A, 7B, 7C and 7D), while the type IIS restriction cleavage site is located on the target complementary portion. It is preferred that the type IIS restriction cleaves 3′ of SNP nucleotide, mutated nucleotide, methylated nucleotide, splicing junction nucleotide, and other specific nucleotide of interest. One of type IIS restriction enzymes may be the Fok I. Fok I restriction enzyme cleaves DNA at any predetermined site with oligodeoxynucleotide adapter-primer which is formed by annealing ART probe and helper primer or extension of helper primer on the ART probe template. The target complementary portion of ART selects a complementary sequence on the target denatured DNA or RNA, hybridizes with it to form the double stranded cleavage site. However before Fok I enzyme can cleave target strand, the single strand Fok I recognition site on ART must be converted to functional double stranded DNA. This is accomplished by helper primer. In some embodiments, the target complementary portions of helper primer and ART probe hybridize to the target at adjacent sites and some portions of helper primer and ART probe hybridize to each other. In some further embodiments, the helper primer and ART probe are designed such that they only anneal to each other in the presence of the specific target. Following helper primer anneals to ART probe in the presence of a target sequence, a DNA polymerase extends the 3′ end of helper primer by copying template portion of ART probe to produce a double stranded functional Fok I recognition site (FIG. 8 and 13).

In some embodiments of the invention, when the target complementary portions of ART hybridizes to free 3′ ends of target DNA or RNA molecules which may be from any source, the 3′ ends of target may be extended by a DNA polymerase using the ART probe as template directly. Therefore, all functional enzyme acting portions are formed without a need of digesting target strand.

In some CELA reactions, high specificity may be achieved by the following factors. First, target specific hybridization between target complementary portions of ART probes and target sequences provides the first level of specificity. Second, the annealing of helper primer to ART probe and/or the extension of 3′ end of helper primer using the probe as template may take place only in the presence of target sequence which bring the ART probe and helper primer together. Third, if occasionally a non-target-specific hybridization occurs, a type IIS restriction enzyme (if used) does not cleave or inefficiently cleave a mismatch at cleavage site so that a chain reaction does not occur. Finally, if non-target-specific hybridization and mismatch cleavage by the type IIS restriction enzyme occur, because of the mismatch nucleotide the 3′ end of nicking site may not be extendable by a DNA polymerase.

C. Treating the Probes Containing Double Stranded Enzyme Acting Portion(s) so as to Produce the Single Stranded End Product (SSEP)

In some embodiments, when the enzyme acting portions of the probe comprise a restriction site, the step (c) comprises: digesting opposite strands of the probes on the restriction site by a restriction enzyme, extending the 3′ end of the digested strand by a DNA polymerase, and repeating digestion and extension, whereby multiple copies of SSEP DNA are produced. In further embodiments, extending the 3′ end of the digested strand may further comprise strand displacing by the DNA polymerase or other strand displacement factors. The suitable DNA polymerases and restriction enzymes are described in the Material Section.

In some embodiments, when the enzyme acting portions of the probe comprise RNA polymerase promoter, the step (c) comprises: repeated transcription by the RNA polymerase acting on the RNA polymerase promoter, whereby multiple copies of SSEP RNA are produced.

In some embodiments, when the enzyme acting portions of the probe comprise both restriction site and RNA polymerase promoter, the step (c) comprises: digesting opposite strands of the probes on the restriction site by a restriction enzyme, extending the 3′ end of digested strands by a DNA polymerase, repeating digestion and extension, whereby multiple copies of SSEP DNA are produced, and repeated transcription by the RNA polymerase acting on the RNA polymerase promoter, whereby multiple copies of SSEP RNA are produced. In further embodiments, extending the 3′ end of the digested strand may further comprise strand displacing by the DNA polymerase or other strand displacement factors.

D. Annealing the SSEP to Free Probes and Causing All Enzyme Acting Portions of Said Probes Double Stranded and Fully Functional

In some embodiments, when the SSEP are DNA molecules or RNA molecules or both DNA and RNA molecules, the step (d) comprises: annealing the SSEP to sequence portions of free probes and extending the 3′ ends of the SSEP using the free probes as templates, whereby all enzyme acting portions of the probes become double stranded and functional. When the probes are linear molecules, the SSEP are complementary to the sequence 5′ to one of the enzyme acting portion (for example the restriction site) of the probe that includes 5′ template portion sequence. Because the 3′ template portion and 5′ template portion of the probe comprise identical or nearly identical sequence, the 3′ end of SSEP is complementary to the 3′ template portion. The SSEP anneal to 3′ template portions of free probes and are extended by the DNA polymerase, therefore double stranded ART probes are formed that trigger repeated digestion and extension. When the probes are circular molecules, the SSEP are complementary to the whole sequence of the ART probe. The SSEP anneal fully or partially to free ART probes and are extended by the DNA polymerase, therefore double stranded ART probes are formed that trigger repeated digestion and extension. It is preferred that extension of the 3′ ends of the SSEP or the 3′ ends of the digested strands may be carried by the DNA polymerase having strand displacement activity or containing other strand displacement factors.

In some embodiments, when the SSEP are RNA molecules, the step (d) comprises: annealing the SSEP to sequence portions of free probes, digesting the SSEP by RNase H, and extending the 3′ end of partially digested SSEP using the free probes as templates, whereby all enzyme acting portions become double stranded and functional.

In some embodiments, when the probes are circular molecules, the sequences of the SSEP comprise one or more than one sequence unit that is complementary to the whole sequence of the probes, step (d) comprises: annealing the SSEP to the whole or parts of the free probes, whereby the enzyme acting portions become double stranded and functional.

If the probes are linear molecule, the SSEP anneal to 3′ template portion of the probe therefore trigger chain reaction. The chance of the SSEP annealing to the 3′ template portions of probes varies, depending on different probe design. Because the SSEP are produced from the 5′ template portion region, the SSEP may anneal to the 5′ template portions of free ART probes more preferably than armeal to 3′ template portions. However, if the 3′ and 5′ template portion sequences are identical, the SSEP may anneal to both portions equally. In any case, a small proportion of SSEP that anneal to 3′ template portion may be sufficient to trigger the chain reaction cascade.

If the probes are circular molecules which comprise only one template portion, the SSEP may comprise one or more full unites that are complementary to the entire circular ART probe. Because of full complementariness of SSEP to the circular ART probe, the SSEP may anneal to free circular ART probes instantly, and may trigger chain reaction efficiently.

E. Repeating Steps (C) and (D), whereby the ART Probes are Converted to Double Stranded or Partially Double Stranded Form, and Multiple Copies of the SSEP are Produced Repeatedly

Because all reagents are in single tube, all above steps may occur simultaneously, there is no distinct boundary between the steps. The step C and D are repeated multiple times. As long as there is free ART probe, the reaction may remain as combined exponential and linear amplification. Once all ART probes are hybridized to SSEP, the linear amplification may be dominant. The reaction accumulates double stranded polynucleotides, single stranded SSEP and PPi that can be labeled and detected.

F. Detection

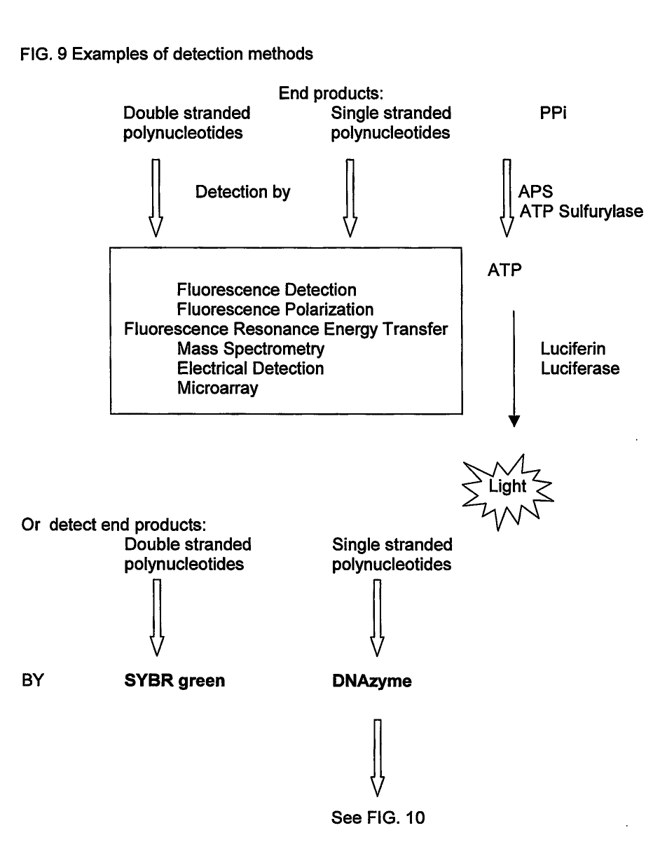

The amplified double stranded end products, single stranded SSEP and PPi can be detected and quantified using any of the conventional detection systems. For examples, the double stranded ARTs and single stranded SSEP can be detected by Fluorescence Detection, Fluorescence Polarization, Fluorescence Resonance Energy Transfer, Mass Spectrometry, Electrical Detection, and Microarray. Specifically, the double stranded ARTs can be detected by binding to a double strand specific fluorescence dye SYBR green. In this invention, the single stranded SSEP is detected by DNAzyme mediated cleavage as outlined below. The PPi generated can be converted to ATP and the resulting ATP concentration is detected and quantified with firefly luciferase (FIG. 9).

CELA products may be detected by incorporating labeled moieties, such as fluorescent nucleotides, biotinylated nucleotides, digoxygenin-containing nucleotides, or bromodeoxyuridine,

In one embodiment for gene expression profiling, a microarray detection system can be used. CELA is multiplexed by using a set of different amplification repeat template (ART) probes, each ART probe carrying different target complementary portions designed for binding to specific target genes, and each ART probe also carrying different template portions designed for SSEP binding to specific oligonucleotides on the microarray. Only those ART probes that are able to hybridize to their targets produce their specific SSEP. In another embodiment for gene expression profiling, ART probes are spotted on microarray. During CELA reaction, double stranded ARTs are created and can be detected and quantified using various detection systems. One of detection system is using SYBR green staining that can be monitored in real time.

G. DNAzyme Mediated Detection of Single Stranded End Products—SSEP

The invention also makes the use of DNAzyme for detection of end products—single stranded SSEP (FIG. 10). The template portions of ART comprise a complementary (antisense) sequence of a DNA enzyme, for example 10-23 DNAzyme. During the CELA reaction, SSEP are produced that contain active (sense) copies of DNAzymes. The DNA enzyme binds an RNA or DNA-RNA chimeric reporter substrate which contain fluorescence resonance energy transfer fluorophores incorporated on either side of a DNAzyme cleavage site. Cleavage of this reporter substrate produces an increase in fluorescence that is indicative of successful CELA amplification.

### EXAMPLE 1

DNA and RNA Isolation. Genomic DNA was isolated from live mouse using standard methods. Total RNA was isolated from mouse thymus and spleen using RNAzol B reagent (Biogenesis Ltd) as recommended by the supplier. Poly(A)+RNA was purified further from total RNA by using a mRNA isolation kit (Qiagen).

Oligonucleotide probe: The ART probe and its target sequence beta-actin gene are illustrated on a map in FIG. 11. The ART probe is 5′dCCGGAGACGTCGTTGTAGCTAGCCTGCGTCsAACAAGCCsGGCTTTGCAC ATGCCGGAGACGTCGTTGp-3′ (SEQ ID NO:1). The italicized nucleotides are the HincII (between 28 to 33) and Nael (between 36 to 41) recognition sites and the underlined nucleotides (15 bases of 3′ and 5′ end) are template sequences. “s” denote phosphorothioate linkage. “p” represents 3′ phosphate. The substrate probe is an RNA/DNA chimeric oligonucleotide 5′-dCCGGAGACGauGCGTCAp-3′ (SEQ ID NO:2), the lower cases is RNA base. 6-carboxyfluorescein (FAM) is incorporated at nucleotide 7 and the quencher 4-(4′-dimethylaminophenylazo)benzoic acid(DABCYL) is incorporated at nucleotide 13 from 5′ end.

CELA reaction conditions were as follows: 50 mM potassium phosphate, pH 7.6, 7.5 mM MgCl2, 8% glycerol, 0.1 mg/ml BSA, 1000 nM ART probe, 200 nM each dATP, dCTP, dGTP and dTTP, 100 units Hincll restriction enzyme (New England Biolabs), 1 unit exo-Klenow (New England Biolabs), 0.1 unit RNase H (Gibco BRL) and the indicated amounts of mouse mRNA. For each sample all reagents except HincII, Klenow and RNase H were assembled in a microcentrifuge tube and the sample was heated at 70 degree C. for 3 min and then equilibrated at room temperature. Then enzymes were added in a single aliquot and reactions were left at room temperature for 5 min. The reactions were then incubated at 37 degree C. for the indicated time. The products were subjected to gel electrophoresis or fluorescence detection.

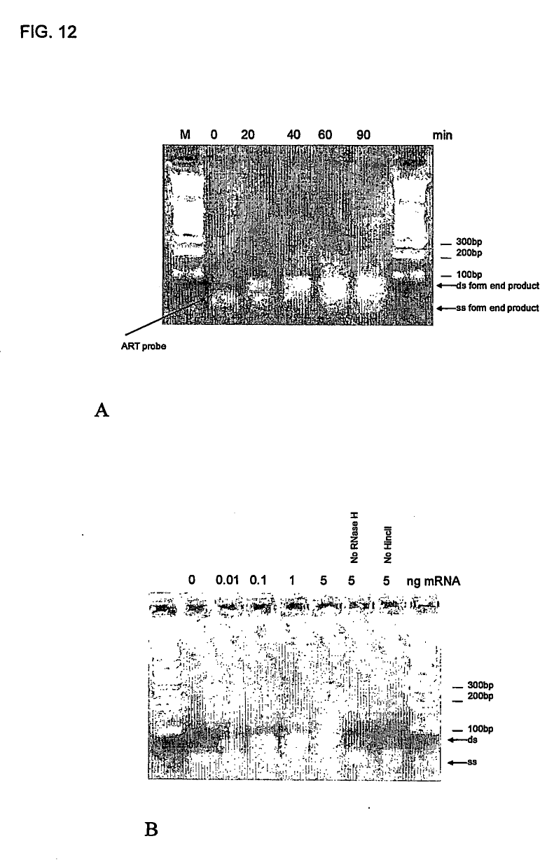

CELA experiments were performed using the same ART probe targeting mouse beta-actin gene from both genomic DNA and mRNA. Samples containing 5 ng mouse mRNA underwent CELA reaction for different time points (FIG. 12A). The double stranded end product can be detected in 20 min, and the single stranded end product is seen in 60 min. Samples containing different amounts of mRNA underwent CELA reaction for 60 min (FIG. 12B). The double stranded product is detected in reaction containing 10 pg mRNA.

One of the end product—single stranded molecule—is a DNAzyme which was detected by catalyzing a substrate (FIG. 11). The DNA enzyme binds DNA-RNA chimeric reporter substrates which contain fluorescence resonance energy transfer fluorophores incorporated on either side of a DNAzyme cleavage site. Cleavage of this reporter substrate produces an increase in fluorescence that is indicative of successful CELA.

### EXAMPLE 2

Oligonucleotide probes: An ART probe, a helper primer and its target sequence are illustrated on a map in FIG. 13. ART probe SNP-G, which corresponds to C allele of p450 target gene, has a sequence of 5′ CCGGAGACGTCGTTGTAGCTAGCCTGCGTCAGGATGCAGCAGCTTsTsCTTG AAGAGCAAACCGGAGACGTCGTTGp 3′ (SEQ ID NO:3). ART probe SNP-A, which corresponds to T allele of p450 target gene, has a sequence of 5′ CCGGAGACGTCGTTGT AGCT AGCCTGCGTCAGGA TGCAGCAGCTTsTsCTT AAAGAGCAAACCGGAGACGTCGTTGp 3′ (SEQ ID NO:4). Synthetic target oligos are: TARGET-C has a sequence of 5′ CCGGTTTGCTCTTCAAGAAAGCTGTGCCCCAGAACACCAGAGp 3 ′ (SEQ ID NO:5); TARGET-G has a sequence of 5′ CCGGTTTGCTCTTTAAGAAAGCTGTGCCCCAGAACACCAGAGp 3′ (SEQ ID NO:6). The helper primer has a sequence of 5′ CTCTGGTGTTCTGGGGCACTGCA 3′ (SEQ ID NO:7). “s” denote phosphorothioate linkage. “p” represents 3′ phosphate.

CELA reaction conditions were as follows: 50 mM potassium phosphate, pH 7.6, 7.5 mM MgCl2, 5% glycerol, 0.1 mg/ml BSA, 1000 nM ART probe, 200 nM each dATP, dCTP, dGTP and dTTP, 50 units Fok I restriction enzyme (New England Biolabs), 1 unit exo-Klenow (New England Biolabs), 10-100 nM helper primer, and the indicated amounts of human DNA or target oligos. For each sample all reagents except Fok I, Klenow were assembled in a microcentrifuge tube and the sample was heated at 95 degree C. or 70 degree C. for 3 min and then equilibrated at room temperature. Then enzymes were added and reactions were left at room temperature for 5 min. The reactions were then incubated at 37 degree C. for the indicated time. The products were subjected to gel electrophoresis or fluorescence detection.

A human DNA sample, which was previously SNP genotyped by other method and is homozygous at C allele of a p450 gene locus, was used in CELA experiments. Gel electrophoresis was able to detect the double stranded end products at 30 min when using 50 ng genomic DNA. Specificity test showed that CELA reactions did not occur when either DNA template or helper primer or Fok I or Klenow was absent in reactions. Synthetic target oligonucleotides were used for sensitivity test, and CELA was able to detect as little as 0.1 amol target in 3 hour reaction. SNP genotyping reactions showed that allele specific probes only reacted with their corresponding targets.

### EXAMPLE 3

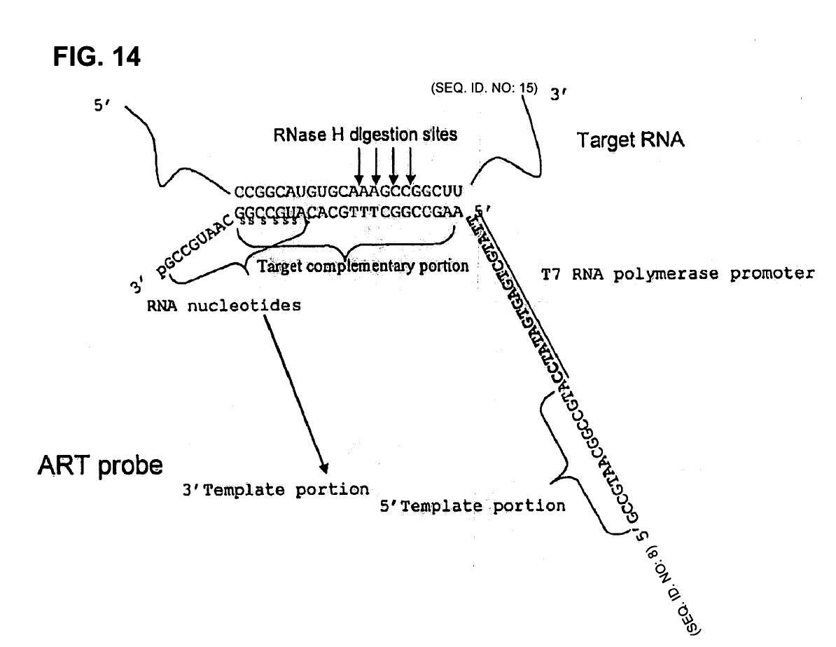

Oligonucleotide probes: An ART probe and its target sequence are illustrated in FIG. 14. ART-T7 has a sequence of 5′ GCCGTAACGGCCGTACCTATAGTGAGTCGTATTAAGCCGGCTTTGCACsAsUs GsCsCsGsGCAAUGCCGp 3′ (SEQ ID NO:8 (T can also be U). The 15 nucleotides at 3′ end (between 49 to 63) are RNA nucleotides. The 18 nucleotides between 16 to 33 is T7 RNA polymerase promoter sequence. The 15 nucleotides at both 3′ and 5′ ends are template portion sequences. The target complementary portion sequence is at between 34 to 55 nucleotides “s” denote phosphorothioate linkage. “p” represents 3′ phosphate.

CELA reaction conditions were as follows: 1× transcription buffer, 1000 nM ART probe, 5 uM each dATP, dCTP, dGTP and dTTP, 2 mM each ATP, CTP, GTP and UTP, 200 units T7 RNA polymerase, 0.1 units RNase H 1 unit exo-Klenow (New England Biolabs), and varying amounts of target RNA. For each sample all reagents except T7 RNA polymerase, Klenowand RNase H were assembled in a microcentrifuge tube and the sample was heated at 70 degree C. for 3 min and then equilibrated at room temperature. Then enzymes were added and reactions were left at room temperature for 5 min. The reactions were then incubated at 37 degree C. for the indicated time. The products were subjected to gel electrophoresis or fluorescence detection.

Total mouse RNA was used as template in CELA experiments. Results showed that CELA specifically occurred when all components were included in reactions, and detected target RNA in 1 ng total RNA sample.

### EXAMPLE 4

Oligonucleotide probes: Circular ART probe was made by hybridization between linear oligonucleotide having a sequence 5′ pGGATGCAGCAGCTTsTsCTTGAAGAGCAAACCGGAGACGTCGTTGTAGCTA G CCTGCGTCA 3′ (SEQ ID NO:9), and a helper primer having a sequence 5′ CTCTGGTGTTCTGGGGCACTGCATCCTGACGCAGAAp 3′ (SEQ ID NO:10), and ligation. All other oligonucleotides are the same as described in Example 2.

CELA reaction conditions were as described in Example 2, and similar experiments were performed. The successful CELA reactions were monitored in gel electrophoresis and in real time detection of fluorescence.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. All publication cited herein are hereby incorporated by reference.

Those skilled in the art will recognize, or be able to ascertain using no more than routine experimentation, many equivalents to the specific embodiments of the invention described herein.

1. A probe molecule comprising single stranded or partially double stranded nucleic acid, wherein said probe comprises: a target complementary portion, a template portion, at least one enzyne acting portion, with or without a 3′ end block portion and wherein said template portion comprises two identical or nearly identical sequences, which are separated by at least one enzyme acting portion when said probe is linear.

2. A probe according to claim 1, wherein said single stranded or partially double stranded nucleic acid is a linear molecule.

3. A probe according to claim 1, wherein said single stranded or partially double stranded nucleic acid is a circular molecule.

4. A probe according to claim 3, wherein said probe is circular probe, wherein said circular probe comprises one template portion.

5. A probe according to claim 1, wherein said enzyme acting portions comprise a RNA polymerase promoter.

6. A probe according to claim 1, wherein said enzyme acting portions comprise RNase H acting sequences.

7. A probe according to claim 1, wherein said enzyme acting portions comprise a nuclease digestion site, wherein said nuclease digestion site support digesting opposite strand of said probe when double stranded.

8. A probe according to claim 1, wherein said at least one enzyme acting portion comprises a restriction enzyme site.

9. A probe according to claim 7, wherein said enzyme acting portions comprise the combination of the RNase H acting sequences and the RNA polymerase promoter or the combination of the RNase H acting sequences and said nuclease digestion sites or the combination of said nuclease digestion sites and the RNA polymerase promoter or the combination of more than one of said nuclease digestion sites.

10. A probe according to claim 7, wherein said nuclease digestion site comprises modified nucleotides, whereby said digestion site on the probe is resistant to nuclease cleavage and the opposite unmodified strand is sensitive to cleavage.

11. A probe according to claim 10, wherein said modified nucleotides comprise phosphorothioate linkages.

12. A probe according to claim 7, wherein said nuclease digestion sites comprise restriction site having a restriction enzyme recognition sequence and a cleavage site.

13. A probe according to claim 12, wherein said restriction site comprises a type IIS restriction enzyme site.

14. A probe according to claim 13, wherein the enzyme cleavage site of said type IIS restriction site is located on target complementary portion.

15. A probe according to claim 14, wherein said type IIS restriction enzyme cleavage site corresponds to a SNP site, mutation nucleotide, methylation nucleotide or splicing site.

16. A probe according to claim 13, wherein said type IIS restriction site is the Fok I site.

17. A probe according to claim 1, comprising helper primer(s), wherein said helper primer comprises at least one portion complementary or substantially complementary to apart of said probe.

18. A probe according to claim 17, wherein said helper primer comprises a 3′ end blocking moiety, whereby the 3′ end of said helper primer is not extendible by a DNA polymerase.

19. A probe according to claim 17, wherein said helper primer does not comprise a 3′ end blocking moiety, whereby the 3′ end of said helper primer is extendible by a DNA polymerase.

20. A probe according to claim 17, wherein said helper primer comprises sequence complementary to the enzyme acting portion(s) with or without flanking sequences or to part of the enzyme acting portion(s) of said probe, whereby hybridization between said helper primer and said probe makes the enzyme acting portion(s) double stranded or partially double stranded.

21. A probe according to claim 17, wherein said helper primer comprises 3′ end sequence complementary to a sequence 3′ to one of the enzyme acting portions of said probe.

22. A probe according to claim 17, wherein said helper primer further comprises target complementary portion(s), wherein the target region(s) complementary to said helper primer is adjacent or substantially adjacent to the target region complementary to said probe.

23. A probe according to claim 22, wherein said helper primer comprises 3′ and 5′ target complementary portions, wherein the target region complementary to said probe is located in the middle of the target regions complementary to said helper primer and is adjacent or substantially adjacent to the target regions complementary to said helper primer.

24. A probe according to claim 1, wherein said target complementary portion comprises sequence complementary or substantially complementary to a target region of interest, whereby said target complementary portion of said probe hybridizes to said target region of interest and becomes double stranded, whereby one or more than one or part of the enzyme acting portion(s) of said probe is partially or fully functional.

25. A probe according to claim 1, wherein said enzyme acting portion(s), said target complementary portion and said template portion(s) of said probe overlap each other or have one portion embedded in other portions.

26. A probe according to claim 1, wherein said target complementary portion and/or said enzyme acting portion(s) and/or said template portion(s) of said probe compose modified nucleotides, whereby modified nucleotides are resistant to nuclease cleavage.

27. A probe according to claim 1, wherein said target complementary portion and/or said enzyme acting portion(s) and/or said template portion(s) of said probe comprise chimeric RNA and DNA.

28. A probe according to claim 1, wherein said probe comprises a catalytically inactive antisense sequence complementary to a DNA enzyme in any place of the circular probe or within the 5′ template portion with or without surrounding portion sequences of the linear probe.

29. A probe according to claim 28, wherein said DNA enzyme is 10-23 DNAzyme.

30. A probe according to claim 28, wherein said DNA enzyme is 8-17 DNAzyme.

31. A probe according to claim 1, wherein said 3′ end block portion is chemical moiety, whereby 3′ end of the probe is not extendible by a DNA polymerase.

32. A probe according to claim 1, wherein any end of said probe and/or helper primer is attached on a solid support.

33. A method of detecting a target nucleic acid sequence or multiple target nucleic acid sequences of interest in a sample, the method comprising the steps of:
(a) contacting probes or a set of probes in accordance with any one of the preceding claims with a nucleic acid sample under suitable hybridization conditions, wherein the target complementary portions of said probes or the target complementary portions of both said probes and helper primers (if present) hybridize the target sequence(s) and become double stranded, whereby one or more than one or part of the enzyme acting portion(s) of said probe is partially or fully functional;
(b) causing all enzyme acting portions of said probes double stranded and fully functional;
(c) treating said probes containing double stranded enzyme acting portion(s) so as to produce the single stranded end product (SSEP);
(d) annealing said SSEP to free probes and causing all enzyme acting portions of said probes double stranded and fully functional, wherein said free probes are the same probes used in step (a);
(e) repeating steps (c) and (d), whereby said probes are converted to double stranded or partially double stranded form, and multiple copies of said SSEP are produced repeatedly, and
(f) detecting directly or indirectly the end products so produced: double stranded end product, SSEP and pyrophosphate (PPi).

34. A method according to claim 33, wherein said method is performed in a single reaction or in separated reactions.

35. A method according to claim 33, wherein said target nucleic acid is RNA and said step (a) causes one of the enzyme acting portion, the RNase H digesting sites, double stranded and functional; wherein said step (b) comprises: digesting RNA strand by RNase H, extending the 3′ end of partially digested strand using said probe as template by a DNA polymerase, whereby all other enzyme acting portions on said probes become double stranded and functional.

36. A method according to claim 35, wherein said extending the 3′ end of partially digested strand further comprises strand displacing by said DNA polymerase or other strand displacement factors.

37. A method according to claim 35, wherein said other enzyme acting portions on said probes comprise restriction site or RNA polymerase promoter or both restriction site and RNA polymerase promoter.

38. A method according to claim 33, wherein one of said enzyme acting portions is restriction site and is located on the target complementary portion of said probe, said step (a) causes said restriction site double stranded and fully functional, wherein said step (b) comprises: digesting opposite strand of said probes on said restriction site by a restriction enzyme, and extending the 3′ end of the digested strand using said probe as template by a DNA polymerase, whereby all other enzyme acting portions on said probes become double stranded and functional.

39. A method according to claim 38, wherein said extending the 3′ end of the digested strand further comprises strand displacing by said DNA polymerase or other strand displacement factors.

40. A method according to claim 38, wherein said other enzyme acting portions on said probes comprise restriction site or RNA polymerase promoter or both restriction site and RNA polymerase promoter.

41. A method according to claim 38, wherein said restriction site is the only enzyme acting portion on said probe.

42. A method according to claim 33, wherein one of said enzyme acting portions is type IIS restriction site, wherein the cleavage site of said type IIS restriction site is located on target complementary portion of said probe and the recognition site of said type IIS restriction site is on either side of target complementary portion of said probe;
wherein step (a) causes the target complementary portions of said probe double stranded, whereby a functional cleavage site of said type IIS restriction site is formed;
wherein said step (b) comprises: annealing helper primers to said probes and causing said recognition sequence of said type IIS restriction site double stranded.

43. A method according to claim 42, wherein said annealing helper primers to said probes and causing said recognition sequence of said type IIS restriction site double stranded comprises: annealing said helper primers directly to said type IIS restriction enzyme recognition sequence with or without flanking sequences whereby double stranded recognition sequence of said type IIS restriction site is formed.

44. A method according to claim 42, wherein said annealing helper primers to said probes and causing said recognition sequence of said type IIS restriction site double stranded comprises: annealing the 3′ end sequence of said helper primer to a sequence 3′ to said type IIS restriction recognition sequence and extending the 3′ end sequence of said helper primer by a DNA polymerase using said probe as template, whereby double stranded recognition sequence of said type IIS restriction site is formed.

45. A method according to claim 33, wherein in said step (a) the target complementary portions of said probes hybridize to free 3′ end(s) of the target sequence(s), said step (b) comprises: extending said free 3′ end(s) of the target sequence(s) by a DNA polymerase using said probes as templates, whereby other enzyme acting portions on said probes become double stranded and functional.

46. A method according to claim 33, wherein said enzyme acting portions of said probe comprise a restriction site, said step (c) comprises: digesting opposite strands of said probes on said restriction site by a restriction enzyme, extending the 3′ end of the digested strand by a DNA polymerase, and repeating said digesting and said extending, whereby multiple copies of SSEP DNA are produced.

47. A method according to claim 46, wherein said extending the 3′ end of the digested strand further comprises strand displacing by said DNA polymerase or other strand displacement factors.

48. A method according to claim 33, wherein said enzyme acting portions of said probe comprise RNA polymerase promoter, said step (c) comprises: repeated transcription by the RNA polymerase acting on said RNA polymerase promoter, whereby multiple copies of SSEP RNA are produced.

49. A method according to claim 33, wherein said enzyme acting portions of said probe comprise both restriction site and RNA polymerase promoter, said step (c) comprises: digesting opposite strands of said probes on said restriction site by a restriction enzyme, extending the 3′ end of digested strands by a DNA polymerase, repeating said digesting and said extending, whereby multiple copies of SSEP DNA are produced, and repeated transcription by the RNA polymerase acting on said RNA polymerase promoter, whereby multiple copies of SSEP RNA are produced.

50. A method according to claim 49, wherein said extending the 3′ end of the digested strand further comprises stand displacing by said DNA polymerase or other strand displacement factors.

51. A method according to claim 33, wherein said SSEP are DNA molecules or RNA molecules or both DNA and RNA molecules, said step (d) comprises: annealing said SSEP to sequence portions of fee probes and extending the 3′ ends of said SSEP using said free probes as templates, whereby all enzyme acting portions of said probes become double stranded and functional.

52. A method according to claim 33, wherein said SSEP are RNA molecules, said step (d) comprises: annealing said SSEP to sequence portions of free probes, digesting said SSEP by RNase H, and extending the 3′ end of partially digested SSEP using said free probes as templates, whereby all enzyme acting portions become double stranded and functional.

53. A method according to claim 33, wherein said probes are circular molecules, the sequences of said SSEP comprise one or more than one sequence unit that is complementary to said probes, step (d) comprises: annealing said SSEP to the whole or parts of said free probes, whereby said enzyme acting portions become double stranded and functional.

54. A method according to claim 33, wherein said template portions comprise antisense DNA enzyme, said method produces multiple copies of single stranded functional sense DNA enzyme, said step (f) of detecting single stranded end product comprises: including a RNA or DNA-RNA chimeric reporter substrate in the reaction, wherein said RNA or DNA-RNA chimeric reporter substrate comprises fluorescence resonance energy transfer fluorophores incorporated on either side of a DNAzyme cleavage site, cleaving said reporter substrate by sense DNA enzyme, whereby cleavage of said reporter substrate produces an increase in fluorescence signal.

55. A kit for use in detecting a target nucleic acid sequence or multiple target nucleic acid sequences of interest in a sample, said kit comprising: said a set or sets of probes as defined in any one of claims 1 to 32, said helper primers, said detection substrate, said restriction enzymes, said RNA polymerase, said RNase H, said DNA polymerase, buffers, dNTPs, NTPs.


SEQUENCE LISTING (16 sequences)

SEQ ID NO: 1 - DNA, 66 bp, Artificial Sequence
  chemcially synthesized
ccggagacgtcgttgtagctagcctgcgtcaacaagccggctttgcacatgccggagacgtcgttg

SEQ ID NO: 2 - DNA, 17 bp, Artificial Sequence
  chemcially synthesized
  RNA (10)..(11)
ccggagacgaugcgtca

SEQ ID NO: 3 - DNA, 74 bp, Artificial Sequence
  chemcially synthesized
ccggagacgtcgttgtagctagcctgcgtcaggatgcagcagctttcttgaagagcaaaccggagacgtcgttg

SEQ ID NO: 4 - DNA, 74 bp, Artificial Sequence
  chemcially synthesized
ccggagacgtcgttgtagctagcctgcgtcaggatgcagcagctttcttaaagagcaaaccggagacgtcgttg

SEQ ID NO: 5 - DNA, 42 bp, Mus musculus
ccggtttgctcttcaagaaagctgtgccccagaacaccagag

SEQ ID NO: 6 - DNA, 42 bp, Mus musculus
ccggtttgctctttaagaaagctgtgccccagaacaccagag

SEQ ID NO: 7 - DNA, 23 bp, Artificial Sequence
  chemcially synthesized
ctctggtgttctggggcactgca

SEQ ID NO: 8 - DNA, 63 bp, Artificial Sequence
  chemically synthesized
  misc_feature (5)..(5): n can be t or u
  misc_feature (14)..(14): n can be t or u
  misc_feature (18)..(18): n can be t or u
  misc_feature (20)..(20): n can be t or u
  misc_feature (23)..(23): n can be t or u
  misc_feature (27)..(27): n can be t or u
  misc_feature (30)..(30): n can be t or u
  misc_feature (32)..(33): n can be t or u
  misc_feature (42)..(44): n can be t or u
  RNA (49)..(63)
gccgnaacggccgnaccnanagngagncgnannaagccggcnnngcacaugccggcaaugccg

SEQ ID NO: 9 - DNA, 59 bp, Artificial Sequence
  chemcially synthesized
ggatgcagcagctttcttgaagagcaaaccggagacgtcgttgtagctagcctgcgtca

SEQ ID NO: 10 - DNA, 36 bp, Artificial Sequence
  chemcially synthesized
ctctggtgttctggggcactgcatcctgacgcagaa

SEQ ID NO: 11 - RNA, 32 nt, Mus musculus
ggcuccggcaugugcaaagccggcuucgcggg

SEQ ID NO: 12 - DNA, 81 bp, Artificial Sequence
  chemcially synthesized
gacgcaggctagctacaacgacgtctccggcatgtgcaaagccggcttgttgacgcaggctagctacaacgacgtctccgg

SEQ ID NO: 13 - DNA, 30 bp, Artificial Sequence
  chemcially synthesized
gacgcaggctagctacaacgacgtctccgg

SEQ ID NO: 14 - DNA, 49 bp, Homo sapiens
ttctggtttgctcttcaagaaagctgtgccccagaacaccagagacctc

SEQ ID NO: 15 - RNA, 22 nt, Mus musculus
ccggcaugugcaaagccggcuu

SEQ ID NO: 16 - DNA, 59 bp, Homo sapiens
ggagacgtcgttgtagctagcctgcgtcaggatgcagcagctttcttgaagagcaaacc